(19) United States
(12) Patent Application Publication
(10) Pub. No.: US 2012/0028985 A1
(43) Pub. Date: Feb. 2, 2012

(54) COMBINATIONAL THERAPY FOR TREATING AUTOIMMUNE DISEASE

(75) Inventors: Manfred Gröppel, Stockdorf (DE); Johann Leban, Planegg-Martinsried (DE)

(73) Assignee: 4SC AG, Planegg-Martinsried (DE)

(21) Appl. No.: 13/128,213

(22) PCT Filed: Nov. 6, 2009
(86) PCT No.: PCT/EP2009/008057

§ 371 Date: Jul. 25, 2011

(30) Foreign Application Priority Data
Nov. 7, 2008 (EP) 08168668.5
Jun. 15, 2009 (EP) 09162716.6

(51) Int. Cl. A61K 31/519; C07C 235/82; C07C 275/42; C07D 307/68; C07D 213/80; A61P 25/00; A61P 37/00; A61P 29/00; A61P 19/02; A61P 17/06; A61P 17/00; A61P 1/00; C07D 333/40; C07D 213/74

(52) U.S. Cl. 514/249; 549/71; 562/455; 562/439; 549/486; 546/310; 546/312; 562/457

(57) ABSTRACT

The present invention relates to the treatment and prevention of immunological and inflammatory disorders with a compound of formula (I) in combination with methotrexate,

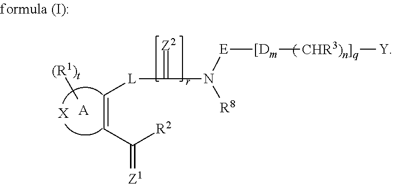

### FIELD OF THE INVENTION

This invention relates to the field of therapeutics more in particular it relates to a pharmaceutical composition. It also relates to a method for treating autoimmune diseases by administration of methotrexate or a pharmaceutically acceptable salt thereof, or a stereoisomer thereof or a tautomer thereof and a compound according to formula (I).

### BACKGROUND OF THE INVENTION

Autoimmune diseases are caused by a misguided immune response regarding parts of the body as foreign. For instance in rheumatoid arthritis parts of the joints are attacked by the immune system, whereas multiple sclerosis is characterized by loss of the myelin sheath due to autoimmune attack.

Rheumatoid arthritis (RA) is an autoimmune disease characterized by chronic inflammation and degeneration of joints. During the course of the disease irreversible joint destruction takes place in addition to extra-articular manifestations. This disorder leads to severe disability and significant reduction in quality of life.

RA is a disease which is quite common especially among the elderly. Its treatment with conventional medications such as non-steroid anti-inflammatory agents is not satisfactory. In view of the increasing aging of the population, especially in the developed Western countries and in Japan the development of new medications for the treatment of RA is urgently required.

In general most cells rely on the salvage pathway to receive the nucleotides required for cell division. But in cases of rapid proliferation this source is not adequate. These rapidly dividing cells have to produce nucleotides de novo. This occurs by two independent pathways for purines and pyrimidines. The most important example for rapid cell proliferation is the clonal expansion of lymphocytes during an immune response. Evidently, a selective inhibitor of this process can be of great therapeutically value for treating RA, and other autoimmune diseases, and for oncological applications.

Dihydroorotate dehydrogenase (DHODH) is the rate limiting enzyme for the de novo synthesis of pyrimidines. This enzyme is a highly promising target for the treatment of the above mentioned diseases.

In fact, various inhibitors have already been identified. Some of them have been clinically tested. The most important example is leflunomide that is currently being used in the routine treatment of rheumatoid arthritis. However, for other compounds like brequinar of FK778 the development has been discontinued in the clinical phase (D V Cramer (1996): Transplant Proc 28, 960 to 963; A Ma and H Chen (2002): Curr Drug Targets Cardiovasc Haematol Disord 2, 57-71).

Experiences of the recent year clearly showed that combination therapy frequently yields improved efficacy compared to monotherapy, especially in early RA. This is in particular true for combinations of disease modifying antirheumatic drugs (DMARDs) with biological agents but also for co-administration of two or more DMARDs (J M Kremer et al. (2002): Ann Intern Med 137, 726-733).

It would thus be advantageous to have a combination therapy at hand that yields improved efficacy compared to monotherapy.

Methotrexate is currently one of the most widely prescribed DMARDs for the treatment of rheumatoid arthritis. Combination therapy of methotrexate with other DMARDs does increase the clinical success of low-dose methotrexate treatment. This has been demonstrated and published e.g. for leflunomide (Ann Intern Med. 2002 Nov. 5; 137(9):142, Clin Exp Rheumatol. 1999 November-December; 17(6 Suppl 18):566-8 and Arthritis Rheum. 1999 July: 42(7)1322-8).

The synergistic effect can be explained by the different and apparently complementary biochemical mechanisms of action of the two compounds (Semin Arthritis Rheum. 1999 August; 29(1):14-26). Low-dose methotrexate inhibits cytokine production, purine biosynthesis, and, in an animal model, caused the release of adenosine, a potent anti-inflammatory agent. Leflunomide, through inhibition of de novo pyrimidine biosynthesis, can regulate lymphocyte proliferation.

The drawback of the combination of methotrexate and leflunomide lies in the toxicity of both compounds to the liver. Severe liver-toxicity has been observed in patients treated with the two compounds (Arthrit. Rheum. 2000; 43(11):2609-11), resulting in the need for careful monitoring in all patients treated with this combination. Therefore, due to the observed additive or even potentiated liver toxicity a combination therapy of methotrexate and leflunomide in RA patients is generally contra-indicated.

Consequently there is a need for safer compositions in this field, displaying less liver-toxicity while maintaining the positive synergistic clinical effect.

### DESCRIPTION OF THE INVENTION

It has now unexpectedly been found that by administering methotrexate in a combination therapy together with DHODH inhibitors of formula (I) as described herein, not only the efficacy of the treatment is increased, but also toxic side effects of methotrexate are diminished. This particular effect is not observed when leflunomide is applied in combination with methotrexate, where actually a potentiation of toxic side effects is observed. This surprising result indicates that liver toxicity caused by leflunomide is a compound-specific effect and that DHODH inhibition in general is not mandatorily linked to liver toxicity as demonstrated by a compound of formula (I).

In a first aspect this invention relates to a kit comprising a first pharmaceutical composition comprising a compound according to formula (I) or a pharmaceutically acceptable salt of formula (I), or a prodrug of formula (I), or a physiologically functional derivative of formula (I), or a stereoisomer of formula (I) or a tautomer of formula (I) and a second pharmaceutical composition comprising methotrexate or a pharmaceutically acceptable salt thereof or a stereoisomer thereof or a tautomer thereof,

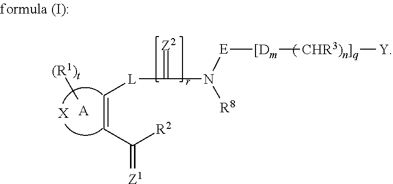

wherein
A is an aromatic or non-aromatic 5- or 6-membered hydrocarbon ring wherein optionally one or more of the carbon atoms are replaced by a group X, wherein X is independently selected from the group consisting of S, O, N, NR⁴, SO₂ and SO;
L is a single bond or NH;
D is O, S, SO₂, NR⁴, or CH₂;
Z¹ is O, S, or NR⁵,
Z² is O, S, or NR⁵;
R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″; cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl;
R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl;
R′ independently represents H, —CO₂R″, —CONR″R′″, —CR″O, —SO₂NR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl, —SO₂-alkynyl, —NR″—CO-alkanyl, —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, -cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;
R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;
R′″ independently represents H or alkanyl;
R² is H or OR⁶, NHR⁷, NR⁷OR⁷;
   or R² together with the nitrogen atom which is attached to R⁸ forms a 5 to 7 membered, preferably 5 or 6 membered heteroyclic ring wherein R² is —[CH₂]ₛ and R⁸ is absent;
R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S-heterocycloalkyl, aralkyl, haloalkanyl, haloalkenyl or haloalkynyl;
R⁴ is H, alkanyl, alkenyl, alkynyl, cycloalkyl, heterocycloalkyl, aryl or heteroaryl;
R⁵ is H, OH, alkanyloxy, alkenyloxy, alkynyloxy, O-aryl, alkanyl, alkenyl, alkynyl or aryl;
R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylalkanyl, (acyloxy)alkanyl, (acyloxy)alkenyl, (acyloxy)alkynyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;
R⁷ is H, OH, alkanyl, alkenyl, alkynyl, aryl, alkanyloxy, alkenyloxy, alkynyloxy, —O-aryl, cycloalkyl, heterocycloalkyl, or —O-cycloalkyl, —O-heterocycloalkyl;
R⁸ is hydrogen, alkanyl, alkenyl or alkynyl;
E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′;
Y is hydrogen, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is

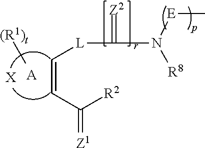

wherein R¹, X, A, Z¹, Z², R⁸, R², E and p are as defined herein;
m is 0 or 1;
n is 0 or 1;
p is 0 or 1;
q is 0 or 1;
r is 0 or 1;
s is 0 to 2; and
t is 0 to 3.

In another preferred embodiment, the present invention relates to a compound of formula (I), wherein
A is an aromatic or non-aromatic 5- or 6-membered hydrocarbon ring wherein optionally one or more of the carbon atoms are replaced by a group X, wherein X is independently selected from the group consisting of S, O, N, NR⁴, SO₂ and SO;
L is a single bond;
D is O, S, SO₂, NR⁴, or CH₂;
Z¹ is O, S, or NR⁵;
Z² is O, S, or NR⁵;
R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″; cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl;
R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl;
R′ independently represents H, —CO₂R″, —CONR″R′″, —CR″O, —SO₂NR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl, —SO₂-alkynyl, —NR″—CO-alkanyl, —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, -cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;
R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;
R′″ independently represents H or alkanyl;
R² is H or OR⁶, NHR⁷, NR⁷OR⁷;
   or R² together with the nitrogen atom which is attached to R⁸ forms a 5 to 7 membered, preferably 5 or 6 membered heteroyclic ring wherein R² is —[CH₂]ₛ and R⁸ is absent;
   R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S-heterocycloalkyl, aralkyl, haloalkanyl, haloalkenyl or haloalkynyl;
R⁴ is H, alkanyl, alkenyl, alkynyl, cycloalkyl, heterocycloalkyl, aryl or heteroaryl;
R⁵ is H, OH, alkanyloxy, alkenyloxy, alkynyloxy, O-aryl, alkanyl, alkenyl, alkynyl or aryl;
R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylmethyl, (acyloxy)alkanyl, (acyloxy)alkenyl, (acyloxy)alkynyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;
R⁷ is H, OH, alkanyl, alkenyl, alkynyl, aryl, alkanyloxy, alkenyloxy, alkynyloxy, —O-aryl, cycloalkyl, heterocycloalkyl, or —O-cycloalkyl, —O-heterocycloalkyl;
R⁸ is hydrogen, alkanyl, alkenyl or alkynyl;
E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′;
Y is hydrogen, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is

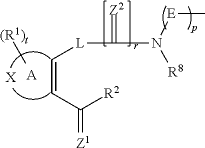

wherein R¹, X, A, Z¹, Z², R⁸, R², E and p are as defined herein;
m is 0 or 1;
n is 0 or 1;
p is 0 or 1;
q is 0 or 1;
r is 0 or 1;
s is 0 to 2; and
t is 0 to 3.

In another preferred embodiment, the present invention relates to a compound of formula (I), wherein
A is an aromatic or non-aromatic 5- or 6-membered hydrocarbon ring wherein optionally one or more of the carbon atoms are replaced by a group X, wherein X is independently selected from the group consisting of S, O, N, N R⁴, SO₂ and SO;
L is NH;
D is O, S, SO₂, NR⁴, or CH₂;
Z¹ is O, S, or NR⁵;
Z² is O, S, or NR⁵;
   R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″; cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl;
R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl;
R′ independently represents H, —CO₂R″, —CONR″R′″, —CR″O, —SO₂NR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl, —SO₂-alkynyl, —NR″—CO-alkanyl, —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, -cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;
R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;
R′″ independently represents H or alkanyl;
R² is H or OR⁶, NHR⁷, NR⁷OR⁷;
   or R² together with the nitrogen atom which is attached to R⁸ forms a 5 to 7 membered, preferably 5 or 6 membered heteroyclic ring wherein R² is —[CH₂]ₛ and R⁸ is absent;
R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S-heterocycloalkyl, aralkyl, haloalkanyl, haloalkenyl or haloalkynyl;
R⁴ is H, alkanyl, alkenyl, alkynyl, cycloalkyl, heterocycloalkyl, aryl or heteroaryl;
R⁵ is H, OH, alkanyloxy, alkenyloxy, alkynyloxy, O-aryl, alkanyl, alkenyl, alkynyl or aryl;
R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylmethyl, (acyloxy)alkanyl, (acyloxy)alkenyl, (acyloxy)alkynyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;
R⁷ is H, OH, alkanyl, alkenyl, alkynyl, aryl, alkanyloxy, alkenyloxy, alkynyloxy, —O-aryl, cycloalkyl, heterocycloalkyl, or —O-cycloalkyl, —O-heterocycloalkyl;
R⁸ is hydrogen, alkanyl, alkenyl or alkynyl;
E is an alkanyl, alkenyl, alkynyl, aryl, heteroaryl, heterocycloalkyl or cycloalkyl group or a fused bi-or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′;
Y is hydrogen, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is

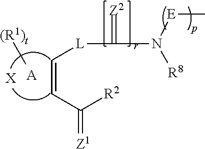

wherein R¹, X, A, Z¹, Z², R⁸, R², E and p are as defined herein;
m is 0 or 1;
n is 0 or 1;
p is 0 or 1;
q is 0 or 1;
r 1;
s is 0 to 2; and
t is 0 to 3.

In another preferred embodiment, the present invention relates to a compound of formula (I), wherein:
A is an aromatic or non-aromatic 5- or 6-membered hydrocarbon ring wherein optionally one or more of the carbon atoms are replaced by a group X, wherein X is independently selected from the group consisting of S, O, N, N R⁴, SO₂ and SO;
L is a single bond or NH;
D is O;
Z¹ is O;
Z² is O;
R¹ independently represents H, halogen, haloalkanyl, haloalkanyloxy, —CO₂R″, —OH, —CN, alkanyloxy, cycloalkyl, heterocycloalkyl, alkanylamino, heteroaryl, alkanyl;
R* independently represents H, alkanyl, R′ independently represents H, cycloalkyl, hydroxyalkanyl, halogen, haloalkanyl, haloalkanyloxy;
R″ independently represents hydrogen, alkanyl;
R² is H or OR⁶;
R³ is H;
R⁴ is H, alkanyl, cycloalkyl;
R⁶ is H, alkanyl;
R⁸ is hydrogen, alkanyl;
E is an alkanyl, aryl, heteroaryl, heterocycloalkyl or cycloalkyl group, wherein all of the aforementioned groups may optionally be substituted by one or more substituents R′;
Y is aryl, heteroaryl, heterocycloalkyl or cycloalkyl group wherein all of the aforementioned groups may optionally be substituted by one or more substituents R′;
   m is 0 or 1;
   n is 0 or 1;
   q is 0 or 1;
   r is 0 or 1;
   and
   t is 0 or 1.

Preferably, in the compounds of Formula (I) L=single bond, and/or Z¹═O (thus r=1), and/or Z²═O, and/or q=0, and/or t=1, and/or R²═OH, and/or R⁸═H.

Preferably, in the compounds of Formula (I) E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring, wherein a carbon is replaced by S.

In a further particularly preferred embodiment, in compounds of formula (I), L=single bond, Z¹═O (thus r=1), Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring, wherein a carbon is replaced by S.

In a further particularly preferred embodiment, in compounds of formula (I), L=single bond, Z¹═O (thus r=1), Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is a non-aromatic hydrocarbon ring, wherein a carbon is replaced by S.

In a further particularly preferred embodiment, in compounds of formula (I), L=single bond, Z¹═O (thus r=1), Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is a non-aromatic hydrocarbon ring.

In a further particularly preferred embodiment, in compounds of formula (I), L=single bond, Z¹═O (thus r=1), Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring, wherein a carbon is replaced by O.

In a further particularly preferred embodiment, in compounds of formula (I), L=NH, Z¹═O (thus r=1), Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring.

In a further particularly preferred embodiment, in compounds of formula (I), L=single bond, r=0, Z²═O, q=0, t=1, R²═OH, R⁸═H and E is heteroaryl which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring.

In a further particularly preferred embodiment, in compounds of formula (I), L=single bond, r=0, Z²═O, q=0, t=1, R²═OH, R⁸═H and E is phenylene which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is an aromatic hydrocarbon ring, wherein a carbon is replaced by N.

In a further particularly preferred embodiment, in compounds of formula (I), L=single bond, Z¹═O (thus r=1), Z²═O, D=O, m=1, n=1, q=1, t=1, R²═OH, R³=H, R⁸═H and E is phenyl which is either unsubstituted or substituted preferably with Cl, F and/or CF₃, OCH₃, OCH₂CH₃, or OCF₃, and Y is phenyl which is also either unsubstituted or substituted preferably with Cl, F and/or CF₃, OCH₃, OCH₂CH₃, or OCF₃, and A is a non-aromatic hydrocarbon ring.

Another further particular embodiment of the compounds of formula (I) according to this invention is a compound of formula (II) or a pharmaceutically acceptable salt, or a prodrug, or a physiologically functional derivative, or a stereoisomer or a tautomer thereof.

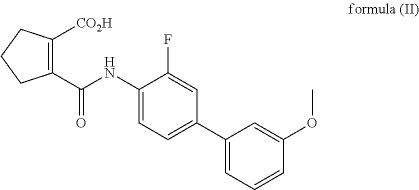

Other further particular embodiments of the compounds of formula (I) according to this invention are compounds selected from the following:
1. 3-(2,3,5,6-Tetrafluoro-3′-trifluoromethoxy-biphenyl-4-ylcarbamoyl)-thiophene-2-carboxylic acid;
2. 4-(2′-Chloro-3,5-difluoro-biphenyl-4-ylcarbamoyl)-2,5-dihydro-thiophene-3-carboxylic acid;
3. 2-[3-Chloro-4-(2-chloro-6-fluoro-benzyloxy)-phenylcarbamoyl]-cyclopent-1-enecarboxylic acid;
4. 2-(2,3,5,6-Tetrafluoro-3′-trifluoromethoxy-biphenyl-4-ylcarbamoyl)-cyclopent-1-enecarboxylic acid;
5. 2-[4-(2-Chloro-6-fluoro-benzyloxy)-3-fluoro-phenylcarbamoyl]-cyclopent-1-enecarboxylic acid;
6. 2-(3-Fluoro-3′-methoxy-biphenyl-4-ylcarbamoyl)-cyclopent-1-enecarboxylic acid;
7. 2-(3-biphenyl-4-ylureido)benzoic acid;
8. 2-(2,3,5,6-tetrafluoro-3′-methoxybiphenyl-4-ylcarbamoyl)furan-3-carboxylic acid;
9. 4-(3′-ethoxy-3,5-difluorobiphenyl-4-ylcarbamoyl)thiophene-3-carboxylic acid;
10. 2-(2,3,5,6-tetrafluoro-3′-methoxybiphenyl-4-ylcarbamoyl)cyclopent-1-enecarboxylic acid;
11. 2-(2,3,5,6-tetrafluoro-2′-methoxybiphenyl-4-ylcarbamoyl)cyclopent-1-enecarboxylic acid;
12. 2-(3,5-difluoro-3′-(trifluoromethoxy)biphenyl-4-ylcarbamoyl)cyclopent-1-enecarboxylic acid;
13. 3-hydroxy-2-(2,3,5,6-tetrafluoro-3′-(trifluoromethoxy)biphenyl-4-ylcarbamoyl)cyclopent-1-enecarboxylic acid;
14. 2-(2-chloro-4′-methoxybiphenyl-4-ylcarbamoyl)cyclopent-1-enecarboxylic acid;
15. 4-(3,5-difluoro-3′-(trifluoromethoxy)biphenyl-4-ylcarbamoyl)thiophene-3-carboxylic acid;
16. 2-[4-(2-Chloro-6-fluoro-benzyloxy)-3-fluoro-phenylcarbamoyl]-cyclopent-1-enecarboxylic acid;
17. 3-(3,5-difluoro-3′-(trifluoromethoxy)biphenyl-4-ylcarbamoyl)thiophene-2-carboxylic acid;
18. 2-(3-fluoro-3′-methoxybiphenyl-4-ylamino)nicotinic acid;
19. 2-(3,5-difluoro-3′-methoxybiphenyl-4-ylamino)nicotinic acid;
20. 5-cyclopropyl-2-(5-methyl-6-(3-(trifluoromethoxy)phenyl)pyridin-3-ylamino)benzoic acid;
21. 2-[4-(2-Chloro-6-fluoro-benzyloxy)-3-fluoro-phenylcarbamoyl]-cyclopent-1-enecarboxylic acid;
22. 2-[3,5-Dichloro-4-(2-chloro-6-fluoro-benzyloxy)-phenylcarbamoyl]-cyclopent-1-enecarboxylic acid;
23. 2-(2-Chloro-4′-dimethylamino-biphenyl-4-ylcarbamoyl)-cyclopent-1-enecarboxylic acid;
24. 3-(3-Fluoro-3′-methoxy-biphenyl-4-ylcarbamoyl)-thiophene-2-carboxylic acid;
25. 4-(2,3,5,6-Tetrafluoro-3′-trifluoromethoxy-biphenyl-4-ylcarbamoyl)-2,5-dihydro-thiophene-3-carboxylic acid;
   or a pharmaceutically acceptable salt, or a prodrug, or a physiologically functional derivative, or a stereoisomer or a tautomer thereof.

An alkanyl group, if not stated otherwise, denotes a linear or branched C₁-C₆-alkanyl, preferably a linear or branched chain of one to five carbon atoms; an alkenyl group, if not stated otherwise, denotes a linear or branched C₂-C₆-alkenyl group comprising one or more carbon-carbon double bonds and which may further comprise one or more carbon-carbon single bonds within its hydrocarbon chain; an alkynyl group, if not stated otherwise, denotes a linear or branched C₂-C₆-alkynyl group comprising one or more carbon-carbon triple bonds and which may further comprise one or more carbon-carbon double and/or single bonds within its hydrocarbon chain, wherein the alkanyl, alkenyl and alkynyl groups can optionally be substituted by one or more substituents R⁹, preferably by halogen.

The C₁-C₆-alkanyl, C₂-C₆-alkenyl and C₂-C₆-alkynyl residue may preferably be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R⁹⁻)₃, —C₂(R⁹)₅, —CH₂—C(R⁹)₃, —C₃(R⁹)₇, —C₂H₄—C(R⁹)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C═C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅, wherein in all of the above-mentioned groups, one or more of the hydrogen atoms can be replaced by a substituent R⁹, preferably by halogen.

R⁹ independently represents H, —CO₂R¹⁰, —CONR¹⁰R¹¹, —CR¹⁰O, —SO₂NR¹⁰, NR¹⁰—CO-haloalkanyl, haloalkenyl, haloalkynyl, —NO₂, —NR¹⁰—SO₂-haloalkanyl, haloalkenyl, haloalkynyl, —NR¹⁰—SO₂-alkanyl, —NR¹⁰—SO₂-alkenyl, —NR¹⁰—SO₂-alkynyl, —SO₂-alkyl, —SO₂-alkenyl, —SO₂-alkynyl, —NR¹⁰—CO-alkanyl, —NR¹⁰—CO-alkenyl, —NR¹⁰—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl.

R¹⁰ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl.

R¹¹ independently represents H or alkanyl.

A cycloalkyl group denotes a monocyclic non-aromatic hydrocarbon ring containing three to eight carbon atoms, preferably four to eight carbon atoms, or a bicyclic non-aromatic hydrocarbon ring system containing seven to ten carbon atoms, preferably eight to ten carbon atoms, wherein the cycloalkyl group optionally comprises one or more double bonds, and wherein the cycloalkyl group is optionally substituted by one or more residues R⁹ as defined above, and wherein in the cycloalkyl group one or two non-consecutive methylene groups may be replaced by a C═O or C═NR⁷ group; non-limiting examples of the cycloalkyl group are cyclopropanyl, cyclobutanyl, cyclopentanyl, cyclohexanyl, cycloheptanyl and cyclooctanyl, preferably cyclopentanyl, cyclohexanyl or cycloheptanyl, wherein in the afore-mentioned groups optionally one or more of the hydrogen atoms is replaced by a residue R⁹ as defined above.

A heterocycloalkyl group denotes a monocyclic non-aromatic hydrocarbon ring containing three to eight carbon atoms, preferably four to eight carbon atoms, or a bicyclic non-aromatic hydrocarbon ring system containing seven to ten carbon atoms, preferably eight to ten carbon atoms, wherein in the heterocycloalkyl group one or more of the carbon atoms of the in the hydrocarbon ring or ring system is replaced by a group selected from the group comprising —N(R⁷)—, —O—, —S—, —S(O)—, —S(O)₂—; wherein the heterocycloalkyl group optionally comprises one or more double bonds, and wherein the heterocycloalkyl group is optionally substituted by one or more residues R′ as defined above, and wherein in the heterocycloalkyl group one or two methylene groups may be replaced by a C═O or C═NR⁷ group; non-limiting examples of the heterocycloalkyl group are azepan-1-yl, piperidinyl, in particular piperidin-1-yl and piperidin-4-yl, piperazinyl, in particular N-piperazinyl and 1-alkylpiperazine-4-yl, morpholine-4-yl, tetrahydrofuranyl, tetrahydrothienyl, pyrrolidinyl, tetrahydropyranyl, tetrahydrothiophen, sulfolanyl, sulfolenyl, oxazolinyl, isoxazolinyl, oxazolidinyl, oxazolidinon-yl, wherein in the afore-mentioned groups optionally one or more of the hydrogen atoms is replaced by a residue R⁹ as defined above.

An alkanyloxy, alkenyloxy or alkynyloxy group denotes an —O-alkanyl, —O-alkenyl or —O-alkynyl group, the alkanyl, alkenyl or alkynyl group being as defined above; the alkanyloxy group is preferably a methoxy, ethoxy, isopropoxy, t-butoxy or pentoxy group.

An alkanylthio, alkenylthio or alkynylthio group denotes an —S-alkanyl, —S-alkenyl or —S-alkynyl group, the alkanyl, alkenyl or alkynyl group being as defined above.

A haloalkanyl, haloalkenyl or haloalkynyl group denotes an alkanyl, alkenyl or alkynyl group which is substituted by one to five halogen atoms, the alkanyl, alkenyl or alkynyl group being as defined above; the haloalkanyl group is preferably a —C(R¹²)₃, —C₂(R¹²)₅, —CH₂—C(R¹²)₃, —CH₂—C(R^{12′})₃, —CH(CH₂(R¹²))₂, —C₃(R¹²)₇ or —C₂H₄—C(R¹²)₃, wherein instances of R¹² may the same or different and each R¹² is independently selected from F, Cl, Br or I, preferably F.

A hydroxyalkanyl, hydroxyalkenyl or hydroxyalkynyl, group denotes an HO-alkanyl, HO-alkenyl or HO-alkynyl group, the alkanyl, alkenyl or alkynyl group being as defined above.

A haloalkanyloxy, haloalkenyloxy or haloalkynyloxy group denotes an alkanyloxy, alkenyloxy or alkynyloxy group which is substituted by one to five halogen atoms, the alkanyl, alkenyl or alkynyl group being as defined above; the haloalkanyloxy, haloalkenyloxy or haloalkynyloxy group is preferably a —OC(R¹²)₃, —OC₂(R¹²)₅, —OCH₂—C(R¹²)₃, —OCH(CH₂(R¹²))₂, —OC₃(R¹²)₇ or —OC₂H₄—C(R¹²)₃, wherein instances of R¹² may the same or different and each R¹² is independently selected from F, Cl, Br or I, preferably F.

A cycloalkyloxy group denotes an —O-cycloalkyl group; the cycloalkynyloxy group is preferably cyclopropoyx, cyclobutoxy and cyclopentoxy.

A hydroxyalkanylamino, hydroxyalkenylamino or hydroxyalkynylamino group denotes an (HO-alkanyl)₂-N—, (HO-alkenyl)₂-N— or (HO-alkynyl)₂-N— group or HO-alkanyl-NH—, HO-alkenyl-NH— or HO-alkynyl-NH— group, the alkanyl, alkenyl or alkynyl group being as defined above.

An alkanylamino, alkenylamino or alkynylamino group denotes an HN-alkanyl, HN-alkenyl or HN-alkynyl or N-dialkanyl, N-dialkenyl or N-dialkynyl group, the alkanyl, alkenyl or alkynyl group being as defined above.

A halogen group is chlorine, bromine, fluorine or iodine, fluorine being preferred.

An aryl group preferably denotes a mono-, bi-, or tricyclic, preferably monocyclic aromatic hydrocarbon group having six to fourteen carbon atoms, wherein the aryl group is optionally substituted by one or more substituents R′, where R′ is as defined above; the aryl group is preferably-o-C₆H₄—R′, -m-C₆H₄—R′, -p-C₆H₄—R′, or phenyl, 1-naphthyl, 2-naphthyl, anthracenyl, in particular 1-anthracenyl and 2-anthracenyl group which may optionally be substituted by one or more R′, more preferably a phenyl group, -o-C₆H₄—R′, -m-C₆H₄—R′, -p-C₆H₄—R′.

A heteroaryl group denotes an aromatic 5-membered monocyclic aromatic hydrocarbon group wherein at least one of the carbon atoms is replaced by a heteroatom like O, N, S, or a- or a 6-membered monocyclic aromatic hydrocarbon group wherein at least one of the carbon atoms is replaced by an N-atom, S, and wherein the aromatic monocyclic 5- or 6-membered cyclic hydrocarbon group is optionally fused to a further monocyclic 5- to 7-membered, preferably 5- or 6-membered, aromatic or non-aromatic hydrocarbon ring, wherein in the further monocyclic aromatic or non-aromatic hydrocarbon ring one or more, preferably one or two carbon atoms may be replaced by a heteroatom like O, N, S; non-limiting examples of heteroaryl groups are thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1-imidazolyl, 2-imidazolyl, 1,2,5-thiadiazol-4-yl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl, 4-pyrazolyl, 1H-tetrazol-2-yl, 1H-tetrazol-3-yl, tetrazolyl, indolyl, indolinyl, benzo[b]furanyl, benzo[b]thiophenyl, benzimidazolyl, benzothiazolyl, quinazolinyl, quinoxazolinyl, or preferably quinolinyl, tetrahydroquinolinyl, isoquinolinyl, tetrahydroisoquinolinyl group; the heteroaryl group can optionally be substituted by one or more substituents R⁹, where R⁹ is as defined above; the skilled person will acknowledge that in the above definition the replacement of a “carbon atom” by a heteroatom includes any hydrogen atoms bound to said carbon atom.

An aralkyl group denotes an aryl group as defined above which is connected to the molecule of the present invention via an alkanyl, alkenyl or alkynyl bridge, wherein alkanyl, alkenyl or alkynyl is as defined above; preferred aralkyl groups are —CH₂—C₆H₅ (benzyl), —CH₂—CH₂—C₆H₅ (phenylethyl), —CH═CH—C₆H₅, —C≡C—C₆H₅, -o-CH₂—C₆H₄—R′, -m-CH₂—C₆H₄—R′, -p-CH₂—C₆H₄—R′; the aralkyl group can optionally be substituted on the aryl and/or alkanyl, alkenyl or alkynyl part by one or more substituents R⁹, wherein R′ is as defined above.

The meaning of E includes alkanyl alkenyl or alkynyl groups optionally substituted by one or more substituents R⁹, wherein alkanyl alkenyl or alkynyl is defined as above and the meaning of E further includes a cycloalkyl group optionally substituted by one or more substituents R⁹ such as cyclopropyl, cyclobutyl, cyclopentyl, cyclohexyl, cycloheptyl, cyclooctyl or carbocyclic aromatic groups such as phenyl, 1-naphthyl, 2-naphthyl, anthracenyl, in particular 1-anthracenyl and 2-anthracenyl, and heteroaromatic groups such as N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 4-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl. E also includes fused polycyclic aromatic ring systems such as 9H-thioxanthene-10,10-dioxide in which a carbocyclic aromatic ring or heteroaryl ring is fused to at least one heteroaryl ring.

The meaning of Y includes is hydrogen, halogen, alkanyl, alkenyl, alkynyl, cycloalkyl or O-aralkyl, wherein all of the aforementioned groups may optionally be substituted with one or more R′ as defined herein, or alternatively Y is E or —O-E, wherein E is as defined herein; in the aforementioned groups, it is furthermore preferred that optional substituents R′ are halogen. Y can also be

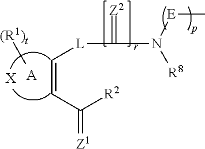

wherein A, X, R¹, R², R⁸, Z¹, Z² and p have the meaning as defined above.

### Specific Embodiments

In certain embodiments A is a 5-membered aromatic hydrocarbon ring wherein one or more, preferably one or two of the carbon atoms are replaced by a group X, wherein X is independently selected from the group consisting of S, O, N or NR⁴.

In certain embodiments A is selected from the group comprising the following:

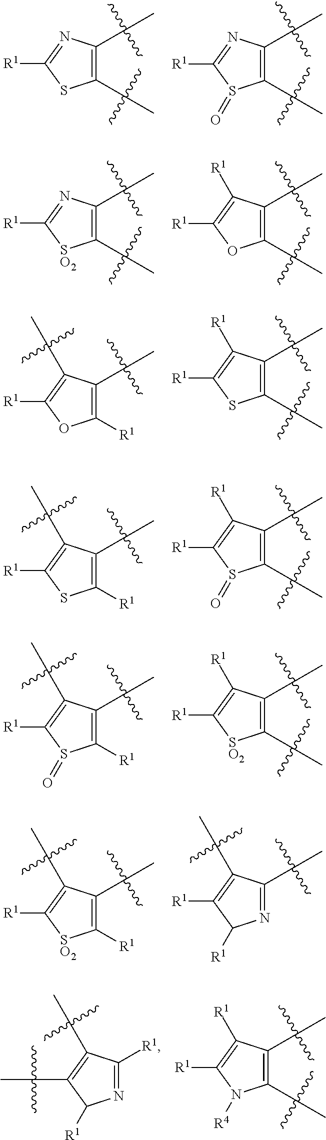

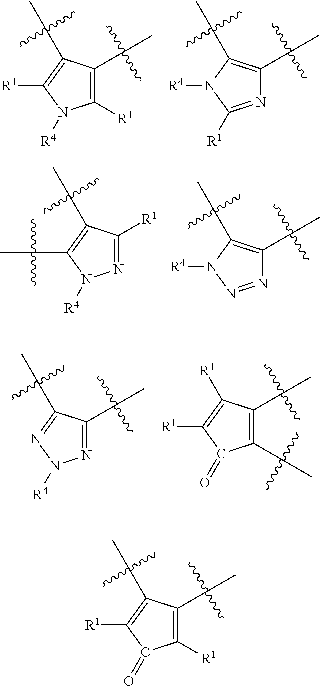

wherein R¹ and R⁴ are defined as above.

In certain embodiments A is a 6-membered aromatic hydrocarbon ring wherein one or more, preferably one or two of the carbon atoms are replaced by a group X, wherein X is independently selected from the group consisting of S, O or N.

In certain embodiments A is selected from the group comprising the following:

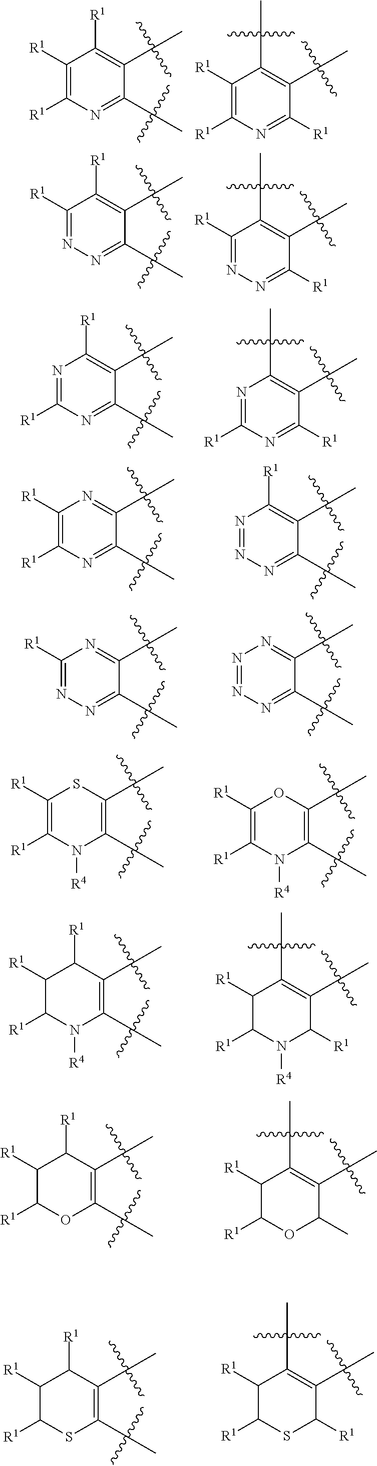

wherein R¹ and R⁴ are defined as above.

In certain embodiments R¹ is H, OH, alkanyl, cycloalkyl, halogen, haloalkanyl CO₂H or SO₃H or tetrazole.

In certain embodiments R² is OH, NH₂, NHOH, NHR⁷, NR⁷OR⁷ or OR⁶.

In certain embodiments R⁶ is benzoyloxymethyl, isobutyryloxymethyl, 4-aminobutyryloxymethyl, butyryloxymethyl, 1-(butyryloxy)ethyl, 1-(butyryloxy)-2,2-dimethylpropyl, 1-diethylphosphonooxyethyl, 2-(2-methoxyethoxy)-acetyloxymethyl, p-aminobenzoylmethyl, nicotinyloxymethyl, pivalyloxymethyl, glutaryloxymethyl, [2-(2-methoxyethoxy)ethoxy]-acetyloxymethyl, 2-(morpholine-4-yl)-ethyl, 1-diethylphosphonooxymethyl.

In certain embodiments R³ is H, alkanyl, alkenyl, alkynyl, cycloalkyl, aryl, alkanyloxy, alkenyloxy, alkynyloxy, O-aryl; O-cycloalkyl, halogen, aminoalkanyl, aminoalkenyl, aminoalkynyl, akanylamino, akenylamino, akynylamino, hydroxylamino, haloalkanyl, haloalkenyl, haloalkynyl, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, S-aryl; S-cycloalkyl, aralkyl, preferably H.

In certain embodiments R⁴ is H, alkanyl, alkenyl, alkynyl, cycloalkyl, aryl or heteroaryl, preferably H.

In certain embodiments R⁸ is H or alkanyl, alkenyl, alkynyl, preferably H or methyl.

In certain embodiments Z¹ and Z² are both O.

In certain embodiments Y is hydrogen, halogen, alkanyl, alkenyl, alkynyl, aryl, heteroaryl, cycloalkyl, heterocycloalkyl or O-aralkyl, wherein all of the aforementioned groups may optionally be substituted with one or more R⁹ as defined herein, or alternatively Y is E or —O-E, wherein E is as defined herein; in the aforementioned groups, it is furthermore preferred that optional substituents R⁹ are halogen. Y can also be:

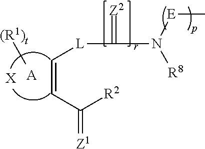

wherein A, X, R¹, R², R⁸, Z¹, Z² and p have the meaning as defined above. Preferably Y is E as defined herein below and more preferably Y is an optionally substituted phenyl.

In certain embodiments, the fused bi- or tricyclic ring system is a bicyclic ring system wherein one phenyl ring is fused to a 5- or 6-membered cycloalkyl or heterocycloalkyl ring or alternatively a tricyclic ring system wherein two phenyl rings are fused to a 5- or 6-membered cycloalkyl or heterocycloalkyl ring, wherein in the tricyclic ring system preferably the 5- or 6-membered cycloalkyl or heterocycloalkyl ring is placed between the two phenyl rings, more preferably the tricyclic ring system is 9H-thioxanthene-10,10-dioxide, wherein all of the aforementioned groups are optionally substituted by one or more substituents R⁹.

In certain embodiments E is an alkyl or cycloalkyl group, preferably selected from the group comprising methyl, ethyl, propyl, butyl, cyclopropyl, cyclobutyl, cyclopentyl, cyclohexyl, cycloheptyl or cyclooctyl, wherein all of the aforementioned groups are optionally substituted by one or more substituents R⁹.

In certain embodiments E is an aryl or heteroaryl group selected from the group comprising phenyl, 1-naphthyl, 2-naphthyl, anthracenyl, in particular 1-anthracenyl and 2-anthracenyl, N-imidazolyl, 2-imidazolyl, 2-thienyl, 3-thienyl, 2-furanyl, 3-furanyl, 2-pyridyl, 3-pyridyl, 4-pyridyl, 2-pyrimidyl, 4-pyrimidyl, 2-pyranyl, 3-pyranyl, 4-pyranyl, 3-pyrazolyl, 4-pyrazolyl, 5-pyrazolyl, 2-pyrazinyl, 2-thiazolyl, 4-thiazolyl, 5-thiazolyl, 2-oxazolyl, 4-oxazolyl and 5-oxazolyl, wherein all of the aforementioned groups are optionally substituted by one or more substituents R⁹.

In certain embodiments E is a fused bi- or tricyclic ring system, which is optionally substituted by one or more substituents R⁹, preferably a 9H-thioxanthene-10,10-dioxide group, which is optionally substituted by one or more substituents R⁹.

In certain embodiments R⁹ is selected from the group comprising cyano, nitro, halogen, alkanyloxy, alkenyloxy, alkynyloxy, cycloalkyloxy, haloalkanyl, haloalkenyl, haloalkynyl, haloalkanyloxy, haloalkenyloxy, haloalkynyloxy, cycloalkyl, hetreocycloalkyl, heteroaryl, alkanyl, alkenyl, alkynyl or aryl, preferably R⁹ is Br, F, Cl, CF₃, OCF₃, —CN, cyclopropoxy, cyclobutoxy, isopropoxy, ethoxy or methoxy.

In certain embodiments the heteroaryl group is selected from the group comprising imidazolyl, thienyl, furanyl, pyridyl, pyrimidyl, pyranyl, pyrazolyl, pyrazinyl, thiazolyl, 1H-tetrazol-2-yl, 1H-tetrazol-3-yl, or oxazolyl.

In certain embodiments t is 0, 1 or 2.

In certain embodiments s is 0 or 1.

In certain embodiments m=1 and D is O, S, SO₂, NR⁴, or CH₂, preferably S or O, more preferably O.

In certain embodiments m=0.

In certain embodiments q=0.

In certain embodiments n=0.

In certain embodiments r is 0 or 1.

In certain embodiments L is a single bond.

In certain embodiments q=1, m=1 and n=1, wherein preferably D=O and/or R³═H).

In certain embodiments R⁸═H.

In certain embodiments A is cyclopenten, thiophen, thiaziol or dihydrothiophen.

In certain embodiments —(C═Z¹)—R² is COOH.

In certain embodiments R¹═H.

In certain embodiments Y is hydrogen, halogen, haloalkanyl, haloalkenyl, haloalkynyl, haloalkanyloxy, haloalkenyloxy, haloalkynyloxy, alkanyl, alkenyl, alkynyl, cycloalkyl or E, preferably F, CF₃, OCF₃, or phenyl, optionally substituted by one or more substituents R⁹, more preferably phenyl, optionally substituted by one or more F, Cl, methoxy, CF₃, or OCF₃

In certain embodiments q=1 and n=0 or 1 and m=1 and D is preferably O.

In a preferred embodiment of the kit, the weight ratio of a compound according to formula (I) or a pharmaceutically acceptable salt of formula (I), or a prodrug of formula (I), or a physiologically functional derivative of formula (I), or a stereoisomer of formula (I) or a tautomer of formula (I) in the first pharmaceutical composition, to methotrexate or a pharmaceutically acceptable salt thereof, or a stereoisomer thereof or a tautomer thereof in the second pharmaceutical composition is between 0.05 and 20, preferably between 0.1 and 10, more preferably between 0.2 and 5, even more preferably between 2 and 4 and most preferably between 2.3 and 3.5.

It is also preferred that the content of a compound according to formula (I) or a pharmaceutically acceptable salt of formula (I), or a prodrug of formula (I), or a physiologically functional derivative of formula (I) in the first pharmaceutical composition is between about 2 and 60 mg, preferably between about 5 and 50 mg.

It is further preferred that the content of methotrexate or a pharmaceutically acceptable salt thereof, or a stereoisomer thereof or a tautomer thereof in the second pharmaceutical composition is between about 5 and 30 mg, preferably between about 10 and 25 mg.

In a particularly preferred embodiment of the kit, for each unit of said second pharmaceutical composition seven units of said first pharmaceutical composition are present in the kit.

Said kits may be used for the treatment or prevention of immunological and inflammatory disorders. Said disorder is preferably rheumatoid arthritis, psoriasis, atopic dermatitis, transplant rejection, systemic lupus erythematosus, inflammatory bowel disease, lupus nephritis or multiple sclerosis, most preferably rheumatoid arthritis.

Preferably the first pharmaceutical composition of the kit is administered once daily and the second pharmaceutical composition is administered once weekly.

It is also preferred that both pharmaceutical compositions of the kit are administered orally.

Methotrexate is herein also abbreviated as MTX.

The compositions described herein are administered via any conventional route. The administration may be carried out, for example, orally, intravenously, intraperitoneally, intramuscularly, subcutaneously or transdermally.

The compositions herein described can be administered orally, e.g. in the form of pills, tablets, coated tablets, sugar-coated tablets, hard and soft capsules, powders, granulates, solutions, syrups or suspensions. Administration can also be carried out rectally, e.g. in the form of suppositories, or parentally, e.g. in the form of injections or infusions.

The compositions described herein can be preferably administered transdermally, e.g. in the form of transdermal therapeutic systems (e.g. patches) or topical formulations (e.g. liposomes, crèmes, ointment, lotion, gels, dispersion, suspension, spray, solution). Topical formulations can also be administered via the pulmonary or nasal route.

In another preferred embodiment the pharmaceutical composition according to the invention is administered orally.

In a further aspect the present invention relates to a compound according to formula (I) or a pharmaceutically acceptable salt of formula (I), or a prodrug of formula (I), or a physiologically functional derivative of formula (I), or a stereoisomer of formula (I) or a tautomer of formula (I) for the use in the treatment or prevention of immunological and inflammatory disorders in a patient, wherein the treatment or prevention additionally comprises the application of methotrexate or a pharmaceutically acceptable salt thereof, or a stereoisomer thereof or a tautomer thereof to the patient.

The present invention also relates to the use of a compound according to formula (I) or a pharmaceutically acceptable salt of formula (I), or a prodrug of formula (I), or a physiologically functional derivative of formula (I), or a stereoisomer of formula (I) or a tautomer of formula (I) for the manufacture of a pharmaceutical composition for the treatment or prevention of immunological and inflammatory disorders in a patient, wherein the treatment or prevention additionally comprises the application of methotrexate or a pharmaceutically acceptable salt thereof, or a stereoisomer thereof or a tautomer thereof to the patient.

The invention further pertains to a method of treating or preventing immunological and inflammatory disorders in a patient comprising administering to the patient a therapeutically effective and tolerable amount of a compound of formula (I) simultaneously, sequentially or separately with a therapeutically effective and tolerable amount of methotrexate or a pharmaceutically acceptable salt thereof, or a stereoisomer thereof or a tautomer thereof.

In a preferred embodiment of a compound, the use of a compound and the method of the invention, the a pharmaceutical composition comprising a compound according to formula (I) or a pharmaceutically acceptable salt of formula (I), or a prodrug of formula (I), or a physiologically functional derivative of formula (I), or a stereoisomer of formula (I) or a tautomer of formula (I) is administered once daily.

It is further preferred that the daily dosage of a compound according to formula (I) or a pharmaceutically acceptable salt of formula (I), or a prodrug of formula (I), or a physiologically functional derivative of formula (I), or a stereoisomer of formula (I) or a tautomer of formula (I) is in the range of from about 2 mg to about 60 mg, preferably in the range of from about 5 mg to about 50 mg.

Preferably, the pharmaceutical composition comprising methotrexate or a pharmaceutically acceptable salt thereof, or a stereoisomer thereof or a tautomer thereof is administered once weekly.

In a particularly preferred embodiment, the weekly dosage of methotrexate or a pharmaceutically acceptable salt thereof, or a stereoisomer thereof or a tautomer thereof is in the range of from about 5 mg to about 30 mg, preferably in the range of from about 10 mg to about 25 mg.

It is further preferred that the pharmaceutical composition comprising a compound according to formula (I) or a pharmaceutically acceptable salt of formula (I), or a prodrug of formula (I), or a physiologically functional derivative of formula (I), or a stereoisomer of formula (I) or a tautomer of formula (I) is administered orally.

It is also preferred that the pharmaceutical composition comprising methotrexate or a pharmaceutically acceptable salt thereof, or a stereoisomer thereof or a tautomer thereof is administered orally.

In a preferred embodiment of a compound, the use of a compound and the method of the invention the disorder is rheumatoid arthritis, psoriasis, atopic dermatitis, transplant rejection, inflammatory bowel disease, systemic lupus erythematosus, lupus nephritis or multiple sclerosis, most preferably the disorder is multiple sclerosis or rheumatoid arthritis.

Preferably, the weight ratio of a compound according to formula (I) or a pharmaceutically acceptable salt of formula (I), or a prodrug of formula (I), or a physiologically functional derivative of formula (I), or a stereoisomer of formula (I) or a tautomer of formula (I) in the pharmaceutical composition that is preferably administered daily, to methotrexate or a pharmaceutically acceptable salt thereof, or a stereoisomer thereof or a tautomer thereof in the second pharmaceutical composition that is preferably administered weekly is between 0.05 and 20, preferably between 0.1 and 10, more preferably between 0.2 and 5, even more preferably between 2 and 4 and most preferably between 2.3 and 3.5.

A further embodiment of the invention is the kit, use or compound of the present invention for the treatment or prevention of a disorder in a patient, the disorder is a disorder wherein the administration of methotrexate to a patient is medically indicated. Thus, the combination therapy presented herein may be applied to treat or prevent any disease which may be treated by methotrexate alone or in combination with further pharmaceutical agents than the ones of formula (I) as described herein. Thereby, the liver-toxicity-diminishing effect is obtainable for the treatment or prevention of all of said diseases. This embodiment encompasses, where applicable, all variations described herein in the further embodiments of the kit, use or compound according to the invention, for instance regarding dosage schemes.

Another specific embodiment of the invention is the kit, compound or use according to any of the preceding claims, wherein the decrease of liver enzyme levels (e.g. ALAT) measured in a spectrophotometric assay, by which the liver toxicity is determined, is 20% or higher, compared with liver enzyme levels (e.g. ALAT) measured in a spectrophotometric assay upon administration of a comparable dose of methotrexate alone (e.g. 5 to 30 mg/week), wherein the spectrophotometric assay comprises the following parameters: spectrophotometer (e.g. KONELAB 30i instrument, Thermo Fisher Scientific, 63303 Dreieich, Germany), sample type=serum, sample volume=15 μl, reagent=ALT reagent, reagent volume=115 μl, incubation time=90 sec., measure time=120 sec., result unit=U/L, wavelength (at which absorption is measured)=340 nm.

ALT reagent comprises 0.2 M L-alanine, 2.0 mM α-ketoglutarate, 100 mM phosphate buffer at pH 7.4 and ALT color reagent, the color reagent comprising 1.0 mM 2,4-dinitrophenylhydrazine in 1N Hydrochloric Acid.

In this context, “a comparable dose of methotrexate alone” means that (within measurement accuracy) the same dose of methotrexate (e.g. 5 to 30 mg/week) is compared to the amount of methotrexate administered in a combination therapy.

In certain embodiments of the invention, a compound according to formula (I) or a pharmaceutically acceptable salt of formula (I), or a prodrug of formula (I), or a physiologically functional derivative of formula (I), or a stereoisomer of formula (I) or a tautomer of formula (I) together with methotrexate or a pharmaceutically acceptable salt thereof or a stereoisomer thereof or a tautomer thereof may be comprised in one pharmaceutical composition. These embodiments encompass, where applicable, all variations described herein in the further embodiments of the kit, use or compound according to the invention.

In the context of the invention a compound and the composition and their pharmacologically acceptable salts can be administered to animals, preferably to mammals, and in particular to humans, dogs and chickens as therapeutics per se, as mixtures with one another or in the form of pharmaceutical preparations which allow enteral or parenteral use and which as active constituent contain an effective dose of the composition of the invention, or a salt thereof, in addition to customary pharmaceutically innocuous excipients and additives.

This invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art. Preferably, the terms used herein are defined as described in “A multilingual glossary of biotechnological terms: (IUPAC Recommendations)”, Leuenberger, H. G. W, Nagel, B. and Kölbl, H. eds., Helvetica Chimica Acta 1995; CH-4010 Basel, Switzerland).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the applicant is not entitled to antedate such disclosure by virtue of prior invention.

The terms “treatment”, “treating” or the like are used herein to generally mean obtaining a desired pharmacologic and/or physiologic effect. The effect may be prophylactic in terms of completely or partially preventing the symptoms of a disease or may be therapeutic in terms of a partial or complete cure of a disease. “Treatment” as used herein also covers any treatment of a disease in a mammal, particularly a human.

The synthesis of these compounds is disclosed in U.S. patent application Ser. Nos. 10/193,526 and 10/736,711, which are hereby fully incorporated by reference.

The present invention relates to compositions with salts of a compound according to formula (I). The salts are preferably cations, most preferably selected from the group consisting of ammonia, arginine, benethamine, benzathine, calcium, choline, deanol, diethanolamine, diethylammonium, ethanolamine, ethylendiamine, meglumine, hydrabamine, imidazole, lysine, magnesium, hydroxyethylmorpholine, piperazine, potassium, epolamine, sodium, trolamine, tromethamine and zinc [Handbook of Pharmaceutical Salts, Ed. P. H. Stahl, C. G. Wermuth, Zurich 2002].

A preferred salt of methotrexate is the di-sodium salt of methotrexate.

Preferred compositions described herein may comprise a carrier material or an excipient, including but are not limited to a lipophilic phase (as for example Vaseline, paraffines, triglycerides, waxes, polyalcylsiloxanes) an oil (olive oil, peanut oil, castor oil, triglyceride oil), an emulsifier (as for example lecithin, phosphatidylglyceroles, alkyl alcohols, sodium lauryl sulfate, polysorbats, Cholesterol, sorbitan fatty acid ester, polyoxyethylene fatty acid glycerol and -ester, poloxamers), a preservative (for instance benzalkonium chloride, chlorobutanol, parabene or thiomersal), a flavouring agent, a buffer substance (for example salts of acetic acid, citric acid, boric acid, phosphoric acid, tatric acid, trometamole or trolamine), a solvent (for instance polyethylenglycols, glycerol, ethanol, isopropanol or propyleneglycol), a solubilizer, an agent for achieving a depot effect, a salt for modifying the osmotic pressure, a carrier material for patches (for instance polypropylene, ethylene-vinylacetate-copolymer, polyacrylates, silicon), an antioxidant (for example ascorbate, tocopherol, butylhydroxyanisole, gallic acid esters or butylhydroxytoluol).

Suitable carrier materials or excipients may further include but are not limited to fillers and extenders (for example lactose, sucrose, mannitol, starch, cellulose, calcium hydrogenphosphate, calciumcarbonate), disintegrants (e.g. starch, cross-linked polyvinylpyrrolidone), binders (for example polyvinylpyrrolidone, mannitol, starch, tragacanth, cellulose, carboxymethylcellulose sodium, gelatine), gliders (for instance talcum, calcium behenate, stearic acid or magnesium stearate), wetting agents (for example sorbitol or glycerol), stabilizers (for example polyacrylic acids, bentonite), emulsifiers (for example hypromellose, hydoxypropylcellulose), preservatives (for instance benzalkonium chloride, chlorobutanol, parabene or thiomersal), sweetening flavouring or aromatizing agents, buffer substances (for example salts of acetic acid, citric acid, boric acid, phosphoric acid, tatric acid, trometamole or trolamine), solvents (for instance polyethylenglycols, glycerol, ethanol, isopropanol or propyleneglycol) or solubilizers, agents for achieving a depot effect, salts for modifying the osmotic pressure, or coating agents (for instance methylcellulose, hypromellose, hydoxypropylcellulose, carboxymethylcellulose sodium, ethylcellulose, methylhydroxypropylcellulosephthalate, celluloseacetate-phthalate, polyvinylpyrrolidone or copolymers of methacrylic acid and acrylate) or antioxidants (for example ascorbate, tocopherol, butylhydroxyanisole, gallic acid esters or butylhydroxytoluol).

The following are examples of the compounds according to the present invention:

| Compound | Structure | UPAC name |
|---|---|---|
| 1 | 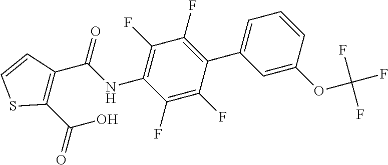 | 3-(2,3,5,6-Tetrafluoro-3′- trifluoromethoxy-biphenyl-4- ylcarbamoyl)-thiophene-2-carboxylic acid |
| 2 | 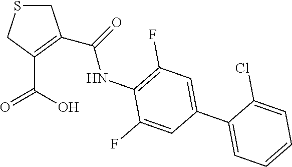 | 4-(2′-Chloro-3,5-difluoro-biphenyl-4- ylcarbamoyl)-2,5-dihydro-thiophene-3- carboxylic acid |
| 3 | 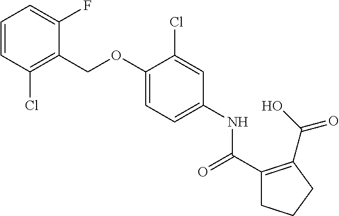 | 2-[3-Chloro-4-(2-chloro-6-fluoro- benzyloxy)-phenylcarbamoyl]- cyclopent-1-enecarboxylic acid |
| 4 | 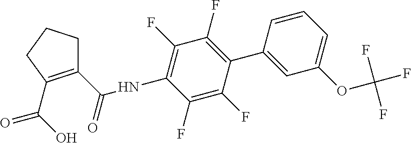 | 2-(2,3,5,6-Tetrafluoro-3′- trifluoromethoxy-biphenyl-4- ylcarbamoyl)-cyclopent-1- enecarboxylic acid |
| 5 | 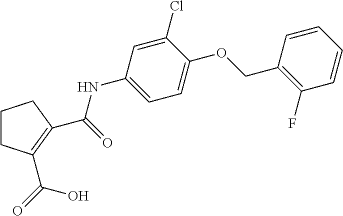 | 2-[4-(2-Chloro-6-fluoro-benzyloxy)-3- fluoro-phenylcarbamoyl]-cyclopent-1- enecarboxylic acid |
| 6 | 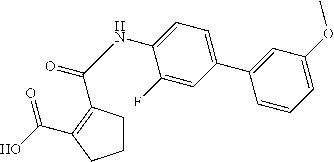 | 2-(3-Fluoro-3′-methoxy-biphenyl-4- ylcarbamoyl)-cyclopent-1- enecarboxylic acid |
| 7 | 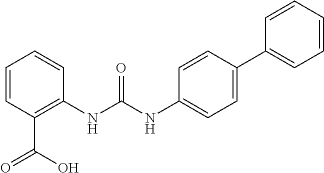 | 2-(3-biphenyl-4-ylureido)benzoic acid |
| 8 | 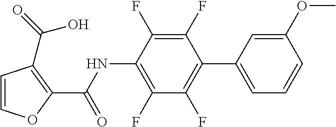 | 2-(2,3,5,6-tetrafluoro-3′- methoxybiphenyl-4- ylcarbamoyl)furan-3-carboxylic acid |
| 9 | 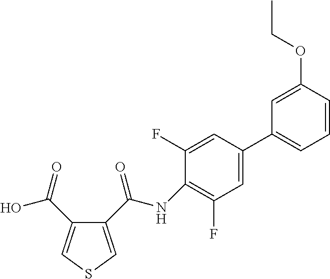 | 4-(3′-ethoxy-3,5-difluorobiphenyl-4- ylcarbamoyl)thiophene-3-carboxylic acid |
| 10 | 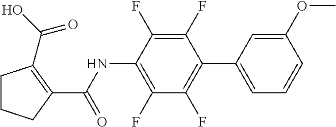 | 2-(2,3,5,6-tetrafluoro-3′- methoxybiphenyl-4- ylcarbamoyl)cyclopent-1- enecarboxylic acid |
| 11 | 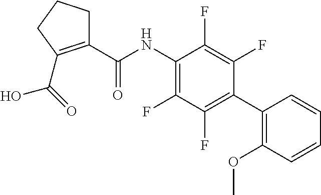 | 2-(2,3,5,6-tetrafluoro-2′- methoxybiphenyl-4- ylcarbamoyl)cyclopent-1- enecarboxylic acid |
| 12 | 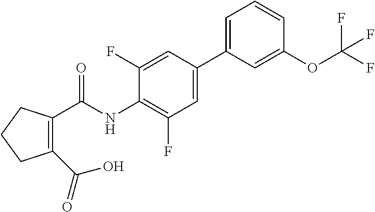 | 2-(3,5-difluoro-3′- (trifluoromethoxy)biphenyl-4- ylcarbamoyl)cyclopent-1- enecarboxylic acid |
| 13 | 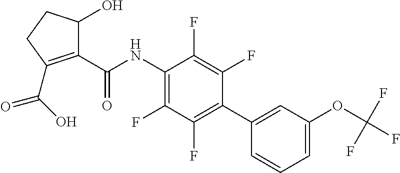 | 3-hydroxy-2-(2,3,5,6-tetrafluoro-3′- (trifluoromethoxy)biphenyl-4- ylcarbamoyl)cyclopent-1- enecarboxylic acid |
| 14 | 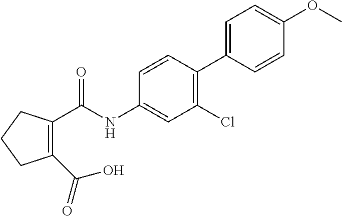 | 2-(2-chloro-4′-methoxybiphenyl-4- ylcarbamoyl)cyclopent-1- enecarboxylic acid |
| 15 | 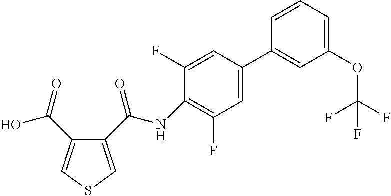 | 4-(3,5-difluoro-3′- (trifluoromethoxy)biphenyl-4- ylcarbamoyl)thiophene-3-carboxylic acid |
| 16 | 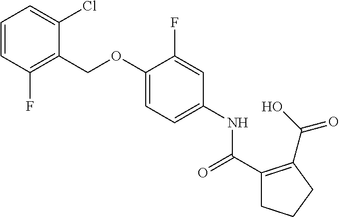 | 2-[4-(2-Chloro-6-fluoro-benzyloxy)-3- fluoro-phenylcarbamoyl]-cyclopent-1- enecarboxylic acid |
| 17 | 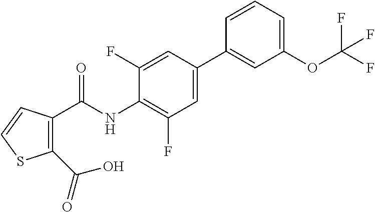 | 3-(3,5-Difluoro-3′-trifluoromethoxy- biphenyl-4-ylcarbamoyl)-thiophene-2- carboxylic acid |
| 18 | 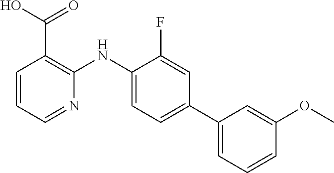 | 2-(3-fluoro-3′-methoxybiphenyl-4- ylamino)nicotinic acid |
| 19 | 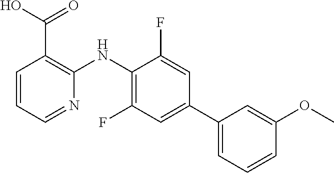 | 2-(3,5-difluoro-3′-methoxybiphenyl-4- ylamino)nicotinic acid |
| 20 | 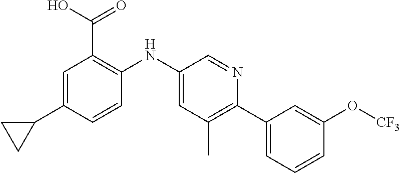 | 5-cyclopropyl-2-(5-methyl-6-(3- (trifluoromethoxy)phenyl)pyridin-3- ylamino)benzoic acid |
| 21 | 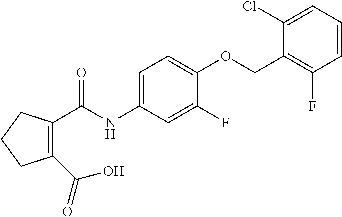 | 2-[4-(2-Chloro-6-fluoro-benzyloxy)-3- fluoro-phenylcarbamoyl]-cyclopent-1- enecarboxylic acid |
| 22 | 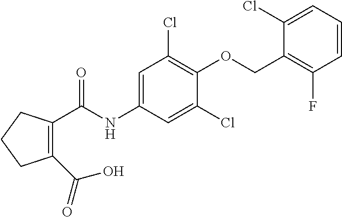 | 2-[3,5-Dichloro-4-(2-chloro-6-fluoro- benzyloxy)-phenylcarbamoyl]- cyclopent-1-enecarboxylic acid |
| 23 | 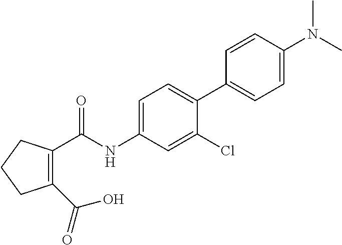 | 2-(2-Chloro-4′-dimethylamino- biphenyl-4-ylcarbamoyl)-cyclopent-1- enecarboxylic acid |
| 24 | 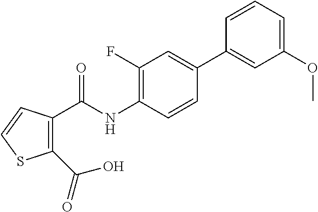 | 3-(3,5-Difluoro-3′-trifluoromethoxy- biphenyl-4-ylcarbamoyl)-thiophene-2- carboxylic acid |
| 25 | 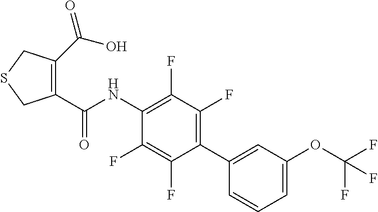 | 4-(2,3,5,6-Tetrafluoro-3′- trifluoromethoxy-biphenyl-4- ylcarbamoyl)-2,5-dihydro-thiophene-3- carboxylic acid |

### FIGURE DESCRIPTION

FIG. 1: Effect of treatment with formula (II)+MTX (methotrexate) on disease development

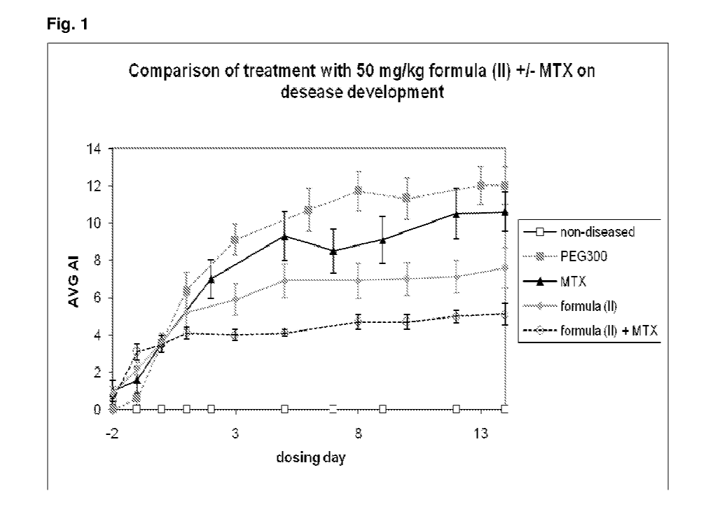

### EXAMPLES

Collagen Induced Arthritis in Mice

Preparation:

Bovine Type II collagen solution is prepared by dissolving at 4 mg/ml in 0.01 M acetic acid at 4-8° C. with stirring overnight. Immunogen is prepared by emulsifying a 1:1 vol:vol combination of collagen solution and Complete Freund's Adjuvant (CFA) (M. tuberculosis H37Ra suspension: 4 mg/ml).

Immunization:

DBA1/J mice (male, 7-8 weeks) are weighed. The animals are anesthetized, injected subcutaneously into the shaved base of the tail with collagen/CFA (0.050 ml/mouse; 100 microgram/mouse collagen in CFA) using a 1 ml syringe fitted with a 25 G needle and returned to the cages.

Boost:

After three weeks (21 d) the procedure is repeated, though now with incomplete Freund's adjuvant (IFA) instead of CFA.

Disease Development:

Animals are observed for one week and macroscopic signs of arthritis are scored 3-times weekly (Monday, Wednesday, Friday: M, W, F). Each paw receives a score:
a) 0=no visible effects of arthritis;
b) 1=edema and/or erythema of 1 digit;
c) 2=edema and/or erythema of 2 digits;
d) 3=edema and/or erythema of more than 2 digits;
e) 4=severe arthritis of entire paw and digits.

Calculate Arthritic Index (AI) by addition of individual paw scores and record. (Maximum AI=16)

After one week, mice are allocated to groups (10 mice per group) to give similar mean AI values and similar ranges of individual AI values in each treatment group.

Dosing:

Dosing is started, PO (per oral) once daily for 14 days.

Treatment Schedule

| | | | |
|---|---|---|---|
| Group No. | | | |
| ROA | No. Mice | Test Material | Dose (mg/kg) |

| | | | | |
|---|---|---|---|---|
| 1 | 10 | PEG300 (vehicle) | N/A | PO |
| | | non-immunized | | |
| | | mice | | |
| 2 | 10 | PEG300 (vehicle) | N/A | PO |
| | | immunized mice | | |
| 3 | 10 | MTX | 2.5 | PO |
| | | Positive Control | | |
| 4 | 10 | formula (II) | 50 | PO |
| 5 | 10 | formula (II) | 50 | PO |
| | | +MTX | 2.5 | PO |

AI scores determined 3× weekly.

Results:

Effect of Disease and Treatment on Arthritic Index

| | | STUDY DAY | | | | | | | | | | | | | |
|---|---|---|---|---|---|---|---|---|---|---|---|---|---|---|---|
| Group | Mouse | 22 | 23 | 24 | 25 | 26 | 27 | 28 | 30 | 33 | 35 | 37 | 40 | 41 | 42 |
| 1 | 1 | 0 | 0 | 0 | 0 | 0 | 0 | 0 | 0 | 0 | 0 | 0 | 0 | 0 | |
| Non- | 2 | 0 | 0 | 0 | 0 | 0 | 0 | 0 | 0 | 0 | 0 | 0 | 0 | 0 | |
| diseased | | | | | | | | | | | | | | | |
| PEG300 po | 3 | 0 | 0 | 0 | 0 | 0 | 0 | 0 | 0 | 0 | 0 | 0 | 0 | 0 | |
| | 4 | 0 | 0 | 0 | 0 | 0 | 0 | 0 | 0 | 0 | 0 | 0 | 0 | 0 | |
| | 5 | 0 | 0 | 0 | 0 | 0 | 0 | 0 | 0 | 0 | 0 | 0 | 0 | 0 | |
| | 6 | 0 | 0 | 0 | 0 | 0 | 0 | 0 | 0 | 0 | 0 | 0 | 0 | 0 | |
| | 7 | 0 | 0 | 0 | 0 | 0 | 0 | 0 | 0 | 0 | 0 | 0 | 0 | 0 | |
| | 8 | 0 | 0 | 0 | 0 | 0 | 0 | 0 | 0 | 0 | 0 | 0 | 0 | 0 | |
| | 9 | 0 | 0 | 0 | 0 | 0 | 0 | 0 | 0 | 0 | 0 | 0 | 0 | 0 | |
| | 10 | 0 | 0 | 0 | 0 | 0 | 0 | 0 | 0 | 0 | 0 | 0 | 0 | 0 | |
| 2 | 11 | 0 | 0 | 0 | 0 | 0 | 6 | 11 | 12 | 12 | 14 | 12 | 12 | 12 | |
| diseased | 12 | 0 | 0 | 0 | 0 | 0 | 6 | 8 | 12 | 16 | 16 | 16 | 16 | 16 | |
| PEG300 | 13 | 0 | 0 | 0 | 0 | 1 | 4 | 8 | 10 | 12 | 11 | 11 | 11 | 11 | |
| po | 14 | 0 | 0 | 0 | 0 | 2 | 3 | 7 | 9 | 9 | 10 | 10 | 9 | 9 | |
| | 15 | 0 | 0 | 0 | 0 | 0 | 2 | 4 | 9 | 12 | 12 | 12 | 12 | 12 | |
| | 16 | 0 | 0 | 0 | 0 | 0 | 3 | 7 | 8 | 10 | 14 | 14 | 16 | 16 | |
| | 17 | 0 | 0 | 0 | 0 | 3 | 5 | 10 | 12 | 16 | 15 | 16 | 16 | 16 | |
| | 18 | 0 | 0 | 0 | 0 | 0 | 2 | 3 | 9 | 8 | 10 | 8 | 8 | 8 | |
| | 19 | 0 | 0 | 0 | 0 | 0 | 2 | 3 | 4 | 5 | 5 | 5 | 8 | 8 | |
| | 20 | 0 | 0 | 0 | 0 | 0 | 3 | 3 | 6 | 7 | 10 | 9 | 12 | 12 | |
| 3 | 21 | 0 | 0 | 0 | 0 | 0 | 1 | 2 | 9 | 11 | 10 | 12 | 12 | | 12 |
| MTX, po | 22 | 0 | 0 | 0 | 0 | 0 | 0 | 2 | 9 | 12 | 10 | 11 | 12 | | 12 |
| 2.5 mg/kg | 23 | 0 | 0 | 0 | 0 | 0 | 1 | 2 | 5 | 8 | 8 | 8 | 9 | | 9 |
| | 24 | 0 | 0 | 0 | 0 | 1 | 1 | 3 | 7 | 9 | 8 | 9 | 9 | | 9 |
| | 25 | 0 | 0 | 0 | 0 | 0 | 1 | 3 | 11 | 12 | 12 | 11 | 14 | | 14 |
| | 26 | 0 | 0 | 0 | 0 | 0 | 0 | 3 | 11 | 14 | 13 | 14 | 14 | | 14 |
| | 27 | 0 | 0 | 0 | 0 | 0 | 0 | 5 | 6 | 11 | 9 | 8 | 12 | | 13 |
| | 28 | 0 | 0 | 0 | 0 | 4 | 6 | 6 | 4 | 4 | 4 | 4 | 5 | | 5 |
| | 29 | 0 | 0 | 0 | 0 | 5 | 6 | 6 | 4 | 5 | 5 | 5 | 6 | | 6 |
| | 30 | 0 | 0 | 0 | 0 | 0 | 0 | 4 | 4 | 7 | 6 | 9 | 12 | | 12 |
| | | STUDY DAY | | | | | | | | | | | | | |
| Group | Mouse | 22 | 23 | 24 | 25 | 26 | 27 | 28 | 29 | 30 | 31 | 32 | 33 | 34 | 35 |
| 4 | 31 | 0 | 0 | 0 | 0 | 0 | 0 | 1 | | 1 | 2 | 5 | 6 | | 5 |
| Formula (II) | 32 | 0 | 0 | 0 | 0 | 0 | 0 | 0 | | 0 | 3 | 6 | 11 | | 12 |
| 50 mg/kg, po | 33 | 0 | 0 | 0 | 0 | 0 | 0 | 0 | | 0 | 1 | 2 | 2 | | 4 |
| | 34 | 0 | 0 | 0 | 0 | 0 | 0 | 1 | | 1 | 1 | 2 | 5 | | 3 |
| | 35 | 0 | 0 | 0 | 0 | 0 | 0 | 0 | | 1 | 3 | 4 | 6 | | 8 |
| | 36 | 0 | 0 | 0 | 0 | 0 | 0 | 0 | | 0 | 3 | 3 | 5 | | 4 |
| | 37 | 0 | 0 | 0 | 0 | 0 | 0 | 1 | | 1 | 1 | 3 | 5 | | 4 |
| | 38 | 0 | 0 | 0 | 0 | 0 | 0 | 2 | | 2 | 2 | 2 | 2 | | 5 |
| | 39 | 0 | 0 | 0 | 0 | 0 | 0 | 2 | | 3 | 4 | 6 | 8 | | 8 |
| | 40 | 0 | 0 | 0 | 0 | 0 | 0 | 0 | | 0 | 2 | 3 | 2 | | 6 |
| 5 | 41 | 0 | 0 | 0 | 0 | 0 | 0 | 0 | 0 | 0 | 0 | 0 | 0 | 0 | 0 |
| Formula (II) | 42 | 0 | 0 | 0 | 0 | 0 | 0 | 0 | 0 | 0 | 0 | 0 | 0 | 0 | 3 |
| 50 mg/kg, po | 43 | 0 | 0 | 0 | 0 | 0 | 0 | 0 | 0 | 0 | 0 | 0 | 0 | 0 | 0 |
| | 44 | 0 | 0 | 0 | 0 | 0 | 0 | 0 | 0 | 0 | 0 | 0 | 0 | 0 | 0 |
| MTX, po | 45 | 0 | 0 | 0 | 0 | 0 | 0 | 0 | 0 | 0 | 0 | 0 | 0 | 0 | 0 |
| 2.5 mg/kg | 46 | 0 | 0 | 0 | 0 | 0 | 0 | 0 | 0 | 0 | 0 | 0 | 0 | 0 | 0 |
| | 47 | 0 | 0 | 0 | 0 | 0 | 0 | 0 | 0 | 0 | 0 | 0 | 0 | 0 | 1 |
| | 48 | 0 | 0 | 0 | 0 | 0 | 0 | 0 | 0 | 0 | 0 | 0 | 0 | 0 | 0 |
| | 49 | 0 | 0 | 0 | 0 | 0 | 0 | 0 | 0 | 0 | 0 | 0 | 0 | 0 | 0 |
| | 50 | 0 | 0 | 0 | 0 | 0 | 0 | 0 | 0 | 0 | 0 | 0 | 0 | 0 | 1 |

STUDY DAY

| Group | Mouse | 37 | 40 | 42 | 44 | 47 |
|---|---|---|---|---|---|---|
| 4 | 31 | 8 | 8 | 8 | 8 | 8 |
| Formula (II) | 32 | 14 | 14 | 14 | 14 | 14 |
| 50 mg/kg, po | 33 | 6 | 6 | 6 | 6 | 10 |
| | 34 | 5 | 4 | 4 | 4 | 3 |
| | 35 | 8 | 8 | 8 | 8 | 12 |
| | 36 | 4 | 4 | 5 | 5 | 5 |
| | 37 | 5 | 5 | 6 | 6 | 6 |
| | 38 | 6 | 6 | 6 | 6 | 7 |
| | 39 | 8 | 8 | 7 | 8 | 6 |
| | 40 | 5 | 6 | 6 | 6 | 5 |

| | | STUDY DAY | | | | | | | | | | | | | |
|---|---|---|---|---|---|---|---|---|---|---|---|---|---|---|---|
| Group | Mouse | 36 | 37 | 38 | 39 | 40 | 41 | 42 | 43 | 44 | 45 | 47 | 49 | 51 | 54 |
| 5 | 41 | 0 | 0 | 2 | 4 | 4 | | 5 | | 4 | | 4 | 4 | 4 | 4 |
| Formula (II) | 42 | 2 | 2 | 3 | 4 | 4 | | 4 | | 4 | | 4 | 4 | 5 | 4 |
| 50 mg/kg, po | 43 | 0 | 0 | 1 | 1 | 2 | | 4 | | 4 | | 4 | 4 | 5 | 5 |
| | 44 | 0 | 0 | 5 | 5 | 5 | | 5 | | 5 | | 5 | 5 | 5 | 3 |
| MTX, po | 45 | 1 | 1 | 4 | 5 | 5 | | 2 | | 4 | | 5 | 5 | 5 | 5 |
| 2.5 mg/kg | 46 | 0 | 0 | 2 | 2 | 3 | | 4 | | 3 | | 5 | 5 | 4 | 5 |
| | 47 | 1 | 1 | 3 | 3 | 4 | | 3 | | 4 | | 4 | 4 | 4 | 4 |
| | 48 | 0 | 0 | 2 | 2 | 5 | | 4 | | 4 | | 4 | 4 | 7 | 8 |
| | 49 | 0 | 0 | 5 | 5 | 4 | | 4 | | 5 | | 4 | 4 | 6 | 8 |
| | 50 | 1 | 1 | 4 | 4 | 5 | | 5 | | 4 | | 8 | 8 | fd | |

Statistical Analysis:

Effect of Disease and Treatment on Average Arthritic Index:

| | | STUDY DAY | | | | | | | | | | | |
|---|---|---|---|---|---|---|---|---|---|---|---|---|---|
| Group | Statistic | 22 | 23 | 24 | 25 | 26 | 27 | 28 | 30 | 31 | 32 | 33 | 34 |
| 1 | Mean | 0 | 0 | 0 | 0 | 0.0 | 0.0 | 0.0 | 0.0 | | | 0.0 | |
| | SD | 0 | 0 | 0 | 0 | 0.0 | 0.0 | 0.0 | 0.0 | | | 0.0 | |
| 2 | Mean | 0 | 0 | 0 | 0 | 0.6 | 3.6 | 6.4 | 9.1 | | | 10.7 | |
| | SD | 0 | 0 | 0 | 0 | 1.1 | 1.6 | 3.0 | 2.6 | | | 3.6 | |
| 3 | Mean | 0 | 0 | 0 | 0 | 1.0 | 1.6 | 3.6 | 7.0 | | | 9.3 | |
| | SD | 0 | 0 | 0 | 0 | 1.8 | 2.3 | 1.6 | 3.3 | | | 4.1 | |
| | p value | | | | | 0.11 | 0.24 | 1.00 | 0.10 | | | 0.38 | |
| 4 | Mean | 0 | 0 | 0 | 0 | 0 | 0 | 0.7 | 0.9 | 2.2 | 3.6 | 5.2 | |
| | SD | 0 | 0 | 0 | 0 | 0 | 0 | 0.8 | 1.0 | 1.0 | 1.6 | 2.9 | |
| | p value | | | | | | | 0.02 | 0.01 | 0.003 | 1.00 | 0.37 | |
| 5 | Mean | 0 | 0 | 0 | 0 | 0 | 0 | 0 | 0 | 0 | 0 | 0 | 0 |
| | SD | 0 | 0 | 0 | 0 | 0 | 0 | 0 | 0 | 0 | 0 | 0 | 0 |
| | p value | | | | | | | | | | | | |

| | | STUDY DAY | | | | | | | | | |
|---|---|---|---|---|---|---|---|---|---|---|---|
| Group | Statistic | 35 | 36 | 37 | 38 | 39 | 40 | 41 | 42 | 43 | 44 |
| 1 | Mean | 0.0 | | 0.0 | | | 0.0 | 0.0 | | | |
| | SD | 0.0 | | 0.0 | | | 0.0 | 0.0 | | | |
| 2 | Mean | 11.7 | | 11.3 | | | 12.0 | 12.0 | | | |
| | SD | 3.2 | | 3.5 | | | 3.2 | 3.2 | | | |
| 3 | Mean | 8.5 | | 9.1 | | | 10.5 | | 10.6 | | |
| | SD | 3.7 | | 4.0 | | | 4.3 | | 3.4 | | |
| | p value | 0.88 | | 0.15 | | | 0.30 | | 0.34 | | |
| 4 | Mean | 5.9 | | 6.9 | | | 6.9 | | 7.0 | | 7.1 |
| | SD | 2.7 | | 2.9 | | | 2.9 | | 2.7 | | 2.8 |
| | p value | 0.004 | | 0.02 | | | 0.01 | | 0.01 | | 0.04 |
| 5 | Mean | 0.5 | 0.5 | 0.5 | | | 4.0 | | 4.1 | | |
| | SD | 1.0 | 0.7 | 0.7 | | | 0.9 | | 0.6 | | |
| | p value | 0.12 | 0.04 | 0.04 | | | 2 × 10⁻⁵ | | 2 × 10⁻⁵ | | |

2. Effect of Disease and Treatment on Average Arthritic Index:

| | | STUDY DAY | | | | | | | |
|---|---|---|---|---|---|---|---|---|---|
| Group | Statistic | 45 | 47 | 49 | 51 | 54 | 56 | 58 | 59 |
| 4 | Mean | | 7.6 | | | | | | |
| | SD | | 3.4 | | | | | | |
| | p value | | 0.01 | | | | | | |
| 5 | Mean | 4.7 | | 4.7 | 5.0 | 5.1 | | | |
| | SD | 1.3 | | 1.3 | 1.0 | 1.8 | | | |
| | p value | 1 × 10⁻⁵ | | 2 × 10⁻⁵ | 1 × 10⁻⁵ | 2 × 10⁻⁵ | | | |

Histological Results:

Examination of the livers from these animals revealed that co-therapy with MTX appeared to prevent the toxicity associated with formula (II) treatment. The data shown indicate that co-administration of formula (II) and MTX has a beneficial effect not only on disease development, but also on liver toxicity in terms of reversing liver toxicity findings caused by administration of formula (II) or MTX alone.

Effect of Disease and Treatment on Liver Histology

| Group* | Mouse | Observations |
|---|---|---|
| 1 | 1 | Normal |
| | 2 | Normal |
| | 3 | Normal |
| | 4 | Normal |
| | 5 | Normal |
| | 6 | Normal |
| | 7 | Normal |
| | 8 | Mild multifocal subacute inflammation |
| | 9 | Normal |
| | 10 | Normal |
| 2 | 11 | Mild glycogen depletion, hepatocyte |
| | 12 | Mild glycogen depletion, hepatocyte |
| | 13 | Mild glycogen depletion, hepatocyte |
| | 14 | Mild glycogen depletion, hepatocyte |
| | 15 | Mild glycogen depletion, hepatocyte |
| | 16 | Mild glycogen depletion, hepatocyte |
| | 17 | Mild glycogen depletion, hepatocyte |
| | 18 | Mild glycogen depletion, hepatocyte |
| | 19 | Mild glycogen depletion, hepatocyte |
| | 20 | Mild glycogen depletion, hepatocyte |
| 3 | 21 | Minimal glycogen depletion, hepatocyte |
| | 22 | Mild glycogen depletion, hepatocyte |
| | 23 | Normal |
| | 24 | Normal |
| | 25 | Mild glycogen depletion, hepatocyte |
| | 26 | Normal |
| | 27 | Mild glycogen depletion, hepatocyte |
| | 28 | Minimal multifocal subacute inflammation |
| | 29 | Mild glycogen depletion, hepatocyte |
| | 30 | Mild glycogen depletion, hepatocyte |
| 4 | 31 | Mild glycogen depletion, hepatocyte |
| | 32 | Mild centrilobular hypertrophy, glycogen |
| | | depletion, hepatocyte |
| | 33 | Mild glycogen depletion, hepatocyte |
| | 34 | Mild glycogen depletion, hepatocyte |
| | 35 | Mild centrilobular hypertrophy, glycogen |
| | | depletion, hepatocyte |
| | 36 | Mild glycogen depletion, hepatocyte |
| | 37 | Mild glycogen depletion, hepatocyte |
| | 38 | Mild glycogen depletion, hepatocyte |
| | 39 | Mild glycogen depletion, hepatocyte |
| | 40 | Mild centrilobular hypertrophy, glycogen |
| | | depletion, hepatocyte |
| 5 | 41 | Normal |
| | 42 | Normal |
| | 43 | Normal |
| | 44 | Mild centrilobular hypertrophy, hepatocyte |
| | 45 | Mild centrilobular hypertrophy, hepatocyte |
| | 46 | Mild centrilobular hypertrophy, hepatocyte |
| | 47 | Mild centrilobular hypertrophy, hepatocyte |
| | 48 | Mild centrilobular hypertrophy, hepatocyte |
| | 49 | Normal |

*Group 1: Non-diseased, PEG300, po
Group 2: diseased, PEG300, po
Group 3: MTX, 2.5 mg/kg, po
Group 4: Formula (formula II),, 50 mg/kg, po
Group 5: Formula (formula II),, 50 mg/kg, po + MTX, 2.5 mg/kg, po

Experimental Design of 13-Week Repeat Dose Toxicity in Beagle Dogs—Combination with Methotrexate

The aim of this study was to obtain information on the interactive toxicity of formula (II) given simultaneously in combination with methotrexate (MTX) by repeated oral administration to Beagle dogs for 13 weeks and to assess the reversibility of any effect at the end of a 4-week recovery period. The animals were randomly allocated to five test groups employing a pseudo-random body weight stratification procedure that yielded groups with approximately equal mean body weights. Formula (II) was administered daily by oral administration, MTX was administered concomitantly once weekly by oral administration. A special focus of the study was to study the influence of the combination treatment on liver toxicity.

TABLE I
Group allocation in combination toxicity study

| | Formula (II) dose | Methotrexate (MTX) dose | |
|---|---|---|---|
| | [mg/kg b.w./day, | [mg/kg b.w./week, | No. and sex of |
| | p.o. | p.o. | animals |
| Group | once daily] | once weekly] | MS + RP |
| 1 | 0 | 0 | 4 + 2 m |
| | (control) | (control) | 4 + 2 f |
| 2 | 25 | 0 | 4 m |
| | (high dose) | (control) | 4 f |
| 3 | 0 | 10/2.5# | 4 m |
| | (control) | | 4 f |
| 4 | 10 | 10/2.5# | 4 m |
| | (low dose) | | 4 f |
| 5 | 25 | 10/2.5# | 4 + 2 m |
| | (high dose) | | 4 + 2 f |

MS Main study
RP Recovery period
m Male
f Female
#The methotrexate dose was reduced to 2.5 mg/kg body weight as of test week 5 for the male animals and test week 4 for the female animals due to a single premature death in group 3.

For laboratory examinations blood samples were taken from the vena cephalica or vena saphena magna from animals fasted overnight. Serum samples were collected into tubes for biochemical tests. Clinical biochemistry parameters from venous blood were determined at the times listed below:

Prior to study start: all animals of a pool of 52 dogs

On test day 37 (approx. 24 hours after 6^{th} Methotrexate administration): all 48 animals included in this study

At main study termination (test day 93): all 48 animals included in this study.

At the end of the recovery period: all 8 recovery animals included in this study

Clinical biochemistry parameters (e.g. bile acids, alanine aminotransferase=ALAT, alkaline phosphatise=aP, aspartate aminotransferase=ASAT, gamma-glutamyl-transferase=Gamma-GT, glutamate dehydrogenase=GLDH, creatine kinase=CK) were measured using KONELAB 30i instrument (Thermo Fisher Scientific, 63303 Dreieich, Germany) as U/L (unit/liter) serum.

Results of 13-Week Repeat Dose Toxicity in Beagle Dogs—Combination with Methotrexate

Oral treatment with 25 mg formula (II)/kg body weight/day alone caused slight signs of systemic intolerance mainly in form of emesis, defecation and soft faeces. One female dog treated with 10 mg MTX/kg body weight/week was found dead in the morning of test day 20. As a result, the MTX dose was reduced to 2.5 mg/kg body weight. Oral treatment with 10/2.5 mg MTX/kg body weight/week alone caused signs of systemic intolerance mainly in form of emesis, defecation and soft faeces.

Oral treatment with 10 or 25 mg formula (II)/kg body weight/day in combination with 10/2.5 mg MTX/kg body weight/week caused increased signs of systemic intolerance mainly in form of emesis, defecation and soft faeces, the intensity of effects caused by the two compounds was additive.

Oral treatment with formula (II) alone revealed no influence on the body weight, food and drinking water consumption and haematological parameters. Treatment with MTX alone or combined treatment with formula (II) and MTX caused comparable reductions in the body weight and food consumption.

Combined treatment of formula (II) and MTX led to several changes in the biochemical parameters identical to MTX alone. In general, oral treatment with formula (II) alone revealed changes in the biochemical parameters in form of slight increased enzyme activities of ALAT, ASAT and GLDH as well as slight increases of the bile acid concentration in serum (GLDH and bile acid data not shown). However, none of these increased values were statistically significant (at p≦0.01). Treatment with MTX alone caused pronounced and statistically significant (at p≦0.01) changes in the biochemical parameters of ALAT, ASAT, CK, GLDH as well as bile acids in comparison to formula (II) alone. For most clinical biochemistry parameters such as ASAT combined treatment with formula (II) and MTX caused similar effects to MTX alone indicating a simple additive toxic response. However, for the key liver enzyme ALAT it has been observed that the combination of MTX and formula (II) caused a significant decrease of ALAT concentration (table II) when compared to MTX or formula (II) given alone indicating a highly beneficial interaction regarding toxic response between these two drugs.

TABLE II
Test item-related changes in selected biochemical parameters
as compared to the control group 1 [%]

| | Group 2 | | | | | | Group 5 | |
|---|---|---|---|---|---|---|---|---|
| | 25 mg | | | | Group 4 | | 25 mg | |
| | Formula | | Group 3 | | 10 mg Formula | | Formula (formula | |
| | (formula | | 10/2.5 mg | | (formula | | II)/kg + | |
| | II)/kg | | MTX/kg | | II)/kg + MTX^{#} | | MTX^{#} | |
| Parameter | males | females | males | females | males | females | males | females |

Test day 37

| | | | | | | | | |
|---|---|---|---|---|---|---|---|---|
| ALAT | none | None | none | +190 | −33 | −8 | −17 | −30 |
| ASAT | none | None | none | +47** | +48 | +78 | +27 | +33 |

Test day 93

| | | | | | | | | |
|---|---|---|---|---|---|---|---|---|
| ALAT | None | None | +386 | +567 | none | None | +47 | +105 |
| ASAT | None | None | +55** | +34** | +62** | +76** | +56** | +44** |

**statistically significant at p ≦ 0.01
^{#}10/2.5 mg MTX/kg

No changes were noted for ECG parameters, blood pressure, at ophthalmological and auditory examination, for the organ weights or at macroscopic examination at any of the tested dose levels. Mild enteritis in form of a lympho-plasmacellular infiltration was observed in the gastro-intestinal mucosa of nearly all test item-treated dogs. The finding was most pronounced in the animals treated with MTX alone compared to the animals treated with formula (II) alone or the combination of formula (II) and MTX.

Mean toxicokinetic parameters of formula (II) and methotrexate after individual and concomitant administration to Beagle dogs are shown in the following table Ill. A clear dose response relationship was noted for formula (II) plasma levels on test day 85, though not on test day 1, possibly caused by the severe toxicity of the 10 mg MTX/kg body weight/week, later reduced to 2.5 MTX/kg body weight/week. No accumulation with time was noted. Methotrexate did not appear to influence the formula (II) absorption and vice versa, therefore, no toxicokinetic interactions were observed.

TABLE III
Mean toxicokinetic parameters of formula (II) and methotrexate after
individual and concomitant administration to Beagle dogs

| | | | | AUC_{0-∞} | | Cₘₐₓ | | t_{1/2} | |
|---|---|---|---|---|---|---|---|---|---|
| | | Dose | Time of | [μg * h/ml] | | [μg/ml] | | [h] | |
| | Group | [mg/kg] | sample | male | female | male | female | male | female |
| Formula (II) | 2 | 25 | day 1 | 289 | 773 | 22.7 | 56.3 | 7.8 | 8.6 |
| | | | week 13 | 601 | 1012 | 56.9 | 66.6 | 6.1 | 11.1 |
| | 4 | 10 | day 1 | 322 | 513 | 30.3 | 43.0 | 6.3 | 7.4 |
| | (+MTX) | | week 13 | 371 | 598²⁾ | 35.7 | 40.5 | 6.3 | 26.7 |
| | 5 | 25 | day 1 | 858 | 413 | 60.0 | 33.1 | 8.4 | 7.6 |
| | (+MTX) | | week 13 | 659 | 1186 | 53.6 | 76.3 | 7.9 | 8.1 |
| Methotrexate | 3 | 10 | day 1 | 6.4 | 5.3 | 1.8 | 1.1 | 1.6 | 2.4 |
| | | 2.5¹⁾ | week 13 | 3.0 | 2.2 | 0.7 | 0.5 | 2.6 | 2.3 |
| | 4 | 10 | day 1 | 6.6 | 7.5 | 1.7 | 1.7 | 1.6 | 3.1 |
| | (+10SC) | 2.5¹⁾ | week 13 | 2.0 | 1.9 | 0.4 | 0.4 | 2.0 | 2.6 |
| | 5 | 10 | day 1 | 5.9 | 5.0 | 1.2 | 1.2 | 2.2 | 2.0 |
| | (+25SC) | 2.5¹⁾ | week 13 | 2.5 | 1.4 | 0.6 | 0.2 | 2.1 | 2.7 |

¹⁾ The dose of methotrexate was reduced from 10 to 2.5 mg/kg as of test week 5 (M) and week 4 (F).
²⁾ AUC₀₋ₜ (+10SC) = in combination with 10 mg/kg formula (II);; (+25SC) = in combination with 25 mg/kg formula (II)

In conclusion, the effects of formula (II) and MTX given simultaneously were simply additive of their individual responses, but not greater than that expected by addition of their individual responses, i.e. no synergistic effects or potentiation was noted regarding increased toxicity. In addition, an increase of the formula (II) dose from 10 to 25 mg/kg body weight/day did not potentiate the toxicity profile. In contrast, the data shown indicate that co-administration of formula (II) and MTX has a beneficial effect on selected liver enzymes such as ALAT in terms of reversing enzyme elevation caused by MTX alone.

1. Kit comprising a first pharmaceutical composition comprising a compound according to formula (I) or a pharmaceutically acceptable salt of formula (I), or a prodrug of formula (I), or a physiologically functional derivative of formula (I), or a stereoisomer of formula (I) or a tautomer of formula (I) and a second pharmaceutical composition comprising methotrexate or a pharmaceutically acceptable salt thereof or a stereoisomer thereof or a tautomer thereof, 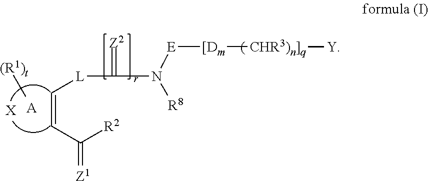
wherein
A is an aromatic or non-aromatic 5-membered hydrocarbon ring wherein optionally one or more of the carbon atoms are replaced by a group X, wherein X is independently selected from the group consisting of S, O, N, NR⁴, SO₂ and SO;
L is a single bond or NH;
D is O, S, SO₂, NR⁴, or CH₂;
Z¹ is O, S, or NR⁵;
Z² is O, S, or NR⁵;
R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″; cycloalkyl, heterocycloalkyl, alkanylamino, alkenylamino, alkynylamino, to hydroxyalkanylamino, hydroxyalkenylamino, hydroxyalkynylamino, —SH, heteroaryl, alkanyl, alkenyl or alkynyl;
R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl;
R′ independently represents H, —CO₂R″, —CONR″R′″, —CR″O, —SO₂NR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl, —SO₂-alkynyl, —NR″—CO-alkanyl, —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, -cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;
R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;
R′″ independently represents H or alkanyl;
R² is H or OR⁶, NHR⁷, NR⁷OR⁷;
or R² together with the nitrogen atom which is attached to R⁸ forms a 5 to 7 membered, preferably 5 or 6 membered heteroyclic ring wherein R² is —[CH₂]ₛ and R⁸ is absent;
R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S-heterocycloalkyl, aralkyl, haloalkanyl, haloalkenyl or haloalkynyl;
R⁴ is H, alkanyl, alkenyl, alkynyl, cycloalkyl, heterocycloalkyl, aryl or heteroaryl;
R⁵ is H, OH, alkanyloxy, alkenyloxy, alkynyloxy, O-aryl, alkanyl, alkenyl, alkynyl or aryl;
R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylalkanyl, (acyloxy)alkanyl, (acyloxy)alkenyl, (acyloxy)alkynyl acyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;
R⁷ is H, OH, alkanyl, alkenyl, alkynyl, aryl, alkanyloxy, alkenyloxy, alkynyloxy, —O-aryl, cycloalkyl, heterocycloalkyl, or —O-cycloalkyl, —O-heterocycloalkyl;
R⁸ is hydrogen, alkanyl, alkenyl or alkynyl;
E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′;
Y is hydrogen, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is 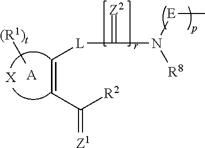
wherein R¹, X, A, Z¹, Z², R⁸, R², E and p are as defined herein;
m is 0 or 1;
n is 0 or 1;
p is 0 or 1;
q is 0 or 1;
r is 0 or 1;
s is 0 to 2; and
t is 0 to 3.

2. Kit according to claim 1, wherein the weight ratio of a compound according to formula (I) or a pharmaceutically acceptable salt of formula (I), or a prodrug of formula (I), or a physiologically functional derivative of formula (I), or a stereoisomer of formula (I) or a tautomer of formula (I) in the first pharmaceutical composition, to methotrexate or a pharmaceutically acceptable salt thereof, or a stereoisomer thereof or a tautomer thereof in the second pharmaceutical composition is between 0.05 and 20, preferably between 0.1 and 10, more preferably between 0.2 and 5, even more preferably between 2 and 4 and most preferably between 2.3 and 3.5.

3. Kit according to claims 1, wherein the content of a compound according to formula (I) or a pharmaceutically acceptable salt of formula (I), or a prodrug of formula (I), or a physiologically functional derivative of formula (I) in the first pharmaceutical composition is between about 2 and 60 mg, preferably between about 5 and 50 mg.

4. Kit according to claim 1, wherein the content of methotrexate or a pharmaceutically acceptable salt thereof, or a stereoisomer thereof or a tautomer thereof in the second pharmaceutical composition is between about 5 and 30 mg, preferably between about 10 and 25 mg.

5. Kit according to claim 1, wherein for each unit of said second pharmaceutical composition seven units of said first pharmaceutical composition are present.

6. A kit according to claim 1, for the treatment or prevention of immunological and inflammatory disorders.

7. A kit according to claim 5, wherein the disorder is rheumatoid arthritis, psoriasis, atopic dermatitis, transplant rejection, systemic lupus erythematosus, inflammatory bowel disease, lupus nephritis or multiple sclerosis, preferably rheumatoid arthritis.

8. Kit according to claim 1, wherein the first pharmaceutical composition is administered once daily and the second pharmaceutical composition is administered once weekly.

9. Kit according to claim 1, wherein both pharmaceutical compositions are administered orally.

10. A Compound according to formula (I) of claim 1, or a pharmaceutically acceptable salt of formula (I), or a prodrug of formula (I), or a physiologically functional derivative of formula (I), or a stereoisomer of formula (I) or a tautomer of formula (I), for the use in the treatment or prevention of immunological and inflammatory disorders in a patient, wherein the treatment or prevention additionally comprises the application of methotrexate or a pharmaceutically acceptable salt thereof, or a stereoisomer thereof or a tautomer thereof to the patient.

11. A method for the treatment or prevention of immunological and inflammatory disorders in a patient, comprising administering to a patient a compound according to formula (I) of claim 1, wherein the treatment or prevention additionally comprises the application of methotrexate or a pharmaceutically acceptable salt thereof, or a stereoisomer thereof or a tautomer thereof to the patient.

12. A compound according to claim 10, wherein a pharmaceutical composition comprising the compound according to formula (I) or a pharmaceutically acceptable salt of formula (I), or a prodrug of formula (I), or a physiologically functional derivative of formula (I), or a stereoisomer of formula (I) or a tautomer of formula (I) is administered once daily.

13. A compound according to claim 12, wherein the daily dosage of the compound according to formula (I) or a pharmaceutically acceptable salt of formula (I), or a prodrug of formula (I), or a physiologically functional derivative of formula (I), or a stereoisomer of formula (I) or a tautomer of formula (I) is in the range of from about 2 mg to about 60 mg, preferably in the range of from about 5 mg to about 50 mg.

14. A compound according to claim 10, wherein a pharmaceutical composition comprising methotrexate or a pharmaceutically acceptable salt thereof, or a stereoisomer thereof or a tautomer thereof is administered once weekly.

15. A compound according to claim 14, wherein the weekly dosage of methotrexate or a pharmaceutically acceptable salt thereof, or a stereoisomer thereof or a tautomer thereof is in the range of from about 5 mg to about 30 mg, preferably in the range of from about 10 mg to about 25 mg.

16. A compound according to claim 10, wherein a pharmaceutical composition comprising the compound according to formula (I) or a pharmaceutically acceptable salt of formula (I), or a prodrug of formula (I), or a physiologically functional derivative of formula (I), or a stereoisomer of formula (I) or a tautomer of formula (I) is administered orally.

17. A compound according to claim 10, wherein a pharmaceutical composition comprising methotrexate or a pharmaceutically acceptable salt thereof, or a stereoisomer thereof or a tautomer thereof is administered orally.

18. A compound according to claim 10, wherein the disorder is rheumatoid arthritis, psoriasis, atopic dermatitis, transplant rejection, inflammatory bowel disease, systemic lupus erythematosus, lupus nephritis or multiple sclerosis.

19. A compound according to claim 18, wherein the disorder is multiple sclerosis or rheumatoid arthritis.

20. The kit according claim 1, wherein liver enzyme levels (e.g. ALAT) measured in a spectrophotometric assay, by which the liver toxicity is determined, are at least 20% reduced compared with liver enzyme levels (e.g. ALAT) measured in a spectrophotometric assay upon administration of a comparable dose of methotrexate alone,
wherein the spectrophotometric assay comprises the following parameters: A spectrophotometer is used, the sample wherein the liver enzyme level is measured is serum, the sample volume is 15 μl, the reagent is ALT reagent, the reagent volume is 115 μl, the incubation, time is 90 sec., the measurement time is 120 sec., the result is quantified in U/L, measurement takes place at a wavelength of 340 nm
